SEQUENCE LISTING (10 sequences)

SEQ ID NO: 1 - DNA, 5543 bp, Bocaparvo virus
  source 1..5543: mol_type = other DNA
gtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccacgtcacatataaaataataaatattcacaaggaggagtggttatatgatgtaatccataaccactcccaggaaatgacgtatgatagccaatcagaattgagtattaaacctatataagctgctgcacttcctgattcaatcagactgcatccggtctccggcgagtgaacatctctggaaaaagctccacgcttgtggtgagtctactatggctttcaatcctcctgtgattagagctttttctcaacctgcttttacttatgtcttcaaatttccatatccacaatggaaagaaaaagaatggctgcttcatgcacttttagctcatggaactgaacaatctatgatacaattaagaaactgcgctcctcatccggatgaagacataatccgtgatgacttgcttatttctttagaagatcgccattttggggctgttctctgcaaggctgtttacatggcaacaactactctcatgtcacacaaacaaaggaatatgtttcctcgttgtgacatcatagttcagtctgagctaggagagaaaaacttacactgccatattatagttgggggagaaggactaagcaagaggaatgctaaatcatcctgtgctcagttctatggtttaatactagctgaaataattcaacgctgcaaatctcttctggctacacgtccttttgaacctgaagaggctgacatatttcacactttaaaaaaggctgagcgagaggcatggggtggagttactggcggcaacatgcaaatccttcaatatagagatcgcagaggagaccttcatgcacaaacagtggatcctcttcgcttcttcaaaaactaccttttacctaaaaatagatgtatttcatcttacagcaaacctgatgtttgtacttctcctgacaactggttcattttagctgaaaaaacttactctcacactcttattaacgggctgccgcttccagaacattacagaaaaaactaccacgcaaccctagataacgaagtcattccagggcctcaaacaatggcctatggaggacgtggtccgtgggaacatcttcctgaggtaggagatcagcgcctagctgcgtcttctgttagcactacttataaacctaacaaaaaagaaaaacttatgctaaacttgctagacaaatgtaaagagctaaatctattagtttatgaagacttagtagctaattgtcctgaactactccttatgcttgaaggtcaaccaggaggggcacgccttatagaacaagtcttgggcatgcaccatattaatgtttgttctaactttacagctctcacatatctttttcatctacatcctgttacttcgcttgactcagacaataaagctttacagcttttgttgattcaaggctataatcctctagccgttggtcacgccctatgctgtgtcctgaacaaacaattcgggaaacaaaacactgtttgcttttacgggcctgcctcaacaggtaaaacaaatatggccaaggcaatcgtccaagggattagactttatgggtgtgttaatcatttgaacaaaggatttgtatttaatgactgcagacaacgcctagttgtttggtgggaggagtgcttaatgcaccaggattgggtggaacctgcaaagtgtatcttgggcgggacagaatgcagaattgacgtcaagcatagagacagtgtacttttaactcaaacacctgtaattatatccactaaccacgatatctacgcggttgttggtggcaattctgtttctcatgttcacgcggctccattaaaagaaagagtgattcagctaaattttatgaaacaacttcctcaaacatttggagaaatcactgctactgagattgcagctcttctacagtggtgtttcaatgagtacgactgtactctgacaggatttaaacaaaaatggaatttagataaaattccaaactcatttcctcttggggtcctttgtcctactcattcacaggactttacacttcacgaaaacggatactgcactgattgcggtggttaccttcctcatagtgctgacaattctatgtacactgatcgcgcaagcgaaactagcacaggagacatcacaccaagtaagtaaatacgcatgcgcaagtaattcttttactttcacttcgctatttttaccaatttttacttttaggtgacttgggggattcggacggagaagacaccaagcctgagacatcgcaagtggactattgtccacccaagaaacgtcgtctaactgctccagcaagtcctccaaactcacctgcgagctctgtaagtactattactttctttaacacttggcacgcacagccacgtgacgaagatgagctcagggaatatgaaagacaagcatcgctcctacaaaagaaaagggagtccagaaagaggggagaggaagagacactggcagacaactcatcacaggagcaggagccgcagcccgatccgacacagtggggagagaggctcgggctcatatcatcaggaacacccaatcagccacctatcgtcttgcactgcttcgaagacctcagaccaagtgatgaagacgagggagagtacatcggggaaaaaagacaatagaacaaatccatacactgtattcagtcaacacagagcttccaatcctgaagctccagggtggtgtgggttctactggcactctactcgcattgctagagatggtactaattcaatctttaatgaaatgaaacaacagtttcaacagctacaaattgataataaaataggatgggataacactagagaactattgtttaatcaaaagaaaacactagatcaaaaatacagaaatatgttctggcactttagaaataactctgattgtgaaagatgtaattactgggatgatgtgtaccgtagacacttagctaatgtttcctcacagacagaagcagacgagataactgacgaggaaatgctttctgctgctgaaagcatggaagcagatgcctccaattaagagacagcctagagggtgggtgctgcctggatacagatatcttgggccatttaatccacttgataacggtgaacctgtaaataacgctgatcgcgctgctcaattacatgatcacgcctactctgaactaataaagagtggtaaaaatccatacctgtatttcaataaagctgatgaaaaattcattgatgatctaaaagacgattggtcaattggtggaattattggatccagtttttttaaaataaagcgcgccgtggctcctgctctgggaaataaagagagagcccaaaaaagacacttttactttgctaactcaaataaaggtgcaaaaaaaacaaaaaaaagtgaacctaaaccaggaacctcaaaaatgtctgacactgacattcaagaccaacaacctgatactgtagacgcaccacagaacacctcagggggaggaacaggaagtattggaggaggaaaaggatctggtgtggggatttccactggagggtgggtcggaggttctcacttttcagacaaatatgtggttactaaaaacacaagacaatttataaccacaattcagaatggtcacctctacaaaacagaggccattgaaacaacaaaccaaagtggaaaatcacagcgctgcgtcacaactccatggacatactttaactttaatcaatacagctgtcacttctcaccacaggattggcagcgccttacaaatgaatataagcgcttcagacctaaagcaatgcaagtaaagatttacaacttgcaaataaaacaaatactttcaaatggtgctgacacaacatacaacaatgacctcacagctggcgttcacatcttttgtgatggagagcatgcttacccaaatgcatctcatccatgggatgaggacgtcatgcctgatcttccatacaagacctggaaactttttcaatatggatatattcctattgaaaatgaactcgcagatcttgatggaaatgcagctggaggcaatgctacagaaaaagcacttctgtatcagatgcctttttttctacttgaaaacagtgaccaccaagtacttagaactggtgagagcactgaatttacttttaactttgactgtgaatgggttaacaatgaaagagcatacattcctcctggactaatgtttaatccaaaagttccaacaagaagagttcagtacataagacaaaacggaagcacagcagccagcacaggcagaattcagccatactcaaaaccaacaagctggatgacaggacctggcctgctcagtgcacagagagtaggaccacagtcatcagacactgctccattcatggtttgcactaacccagaaggaacacacataaacacaggtgctgcaggatttggatctggctttgatcctccaagcggatgtctggcaccaactaacctagaatacaaacttcagtggtaccagacaccagaaggaacaggaaataatggaaacataattgcaaacccatcactctcaatgcttagagaccaactcctatacaaaggaaaccagaccacatacaatctagtgggggacatatggatgtttccaaatcaagtctgggacagatttcctatcaccagagaaaatccaatctggtgcaaaaaaccaagagctgacaaacacacaatcatggatccatttgatggatcaattgcaatggatcatcctccaggcactatttttataaaaatggcaaaaattccagttccaactgcctcaaatgcagactcatacctaaacatatactgtactggacaagtcagctgtgagattgtatgggaagtaaaaagatacgcaacaaagaactggcgtccagaaagaagacatactgcactcgggatgtcactgggaggagaaagcaactacacgcctacataccacgtggatccaacaggagcatacatccagcccacgtcatatgatcaatgtatgccagtaaaaacaaacatcaataaagtgttgtaatcttataagcctcttttttgcttctgcttacaagttcctcctcaatggacaagcggaaagtgaagggtgactgtagtcctgagctcatgggttcaagaccacagcccgatggtagtggtgttaccgtctcgaacctagccgacagcccttgtacattgtggggggagctgttttgtttgcttatgcaatcgcgaaactctatatcttttaatgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcataagcaa

SEQ ID NO: 2 - DNA, 27 bp, synthetic construct
  source 1..27: mol_type = other DNA
ctgtctagaatgatcaatgtatgccag

SEQ ID NO: 3 - DNA, 25 bp, synthetic construct
  source 1..25: mol_type = other DNA
cacggatcctttttttttttttttt

SEQ ID NO: 4 - DNA, 46 bp, synthetic construct
  source 1..46: mol_type = other DNA
cgcgaaactctatatcttttaatggcagaattcagcacatgcgcca

SEQ ID NO: 5 - DNA, 38 bp, synthetic construct
  source 1..38: mol_type = other DNA
gccgccggtcgccgccggtaggcgggacttccggtaca

SEQ ID NO: 6 - DNA, 30 bp, synthetic construct
  source 1..30: mol_type = other DNA
agctattggtcgctattggtaggcgggact

SEQ ID NO: 7 - DNA, 12 bp, synthetic construct
  source 1..12: mol_type = other DNA
tgttgttgttgt

SEQ ID NO: 8-9 - skipped (2 entries)

SEQ ID NO: 10 - DNA, 46 bp, synthetic construct
  source 1..46: mol_type = other DNA
tggcgcatgtacaacaacaacacattaaaagatatagagtttcgcg

(19) United States
(12) Patent Application Publication
(10) Pub. No.: US 2026/0117253 A1
(43) Pub. Date: Apr. 30, 2026

(54) cis AND trans REQUIREMENTS FOR TERMINAL RESOLUTION OF HUMAN BOCAVIRUS 1

(71) Applicants: University of Iowa Resarch Foundation, Iowa City, IA (US); University of Kansas, Lawrence, KS (US)

(72) Inventors: John F. Engelhardt, Iowa City, IA (US); Ziying Yan, Iowa City, IA (US); Jianming Qiu, Overland Park, KS (US)

(21) Appl. No.: 18/943,488

(22) Filed: Nov. 11, 2024

Related U.S. Application Data
(62) Division of application No. 16/304,064, now Pat. No. 12,173,305.
(60) Provisional application No. 62/341,897, filed on May 26, 2016.

(51) Int. Cl. C12N 15/86; C12N 7/00

(52) U.S. Cl. CPC C12N 15/86; C12N 7/00; C12N 2750/14321; C12N 2750/14322; C12N 2750/14343

(57) ABSTRACT

A recombinant mutant BoV genome is provided, as well as methods of using the vector, e.g., to prepare helper-free virus.

### CROSS-REFERENCE TO RELATED APPLICATIONS

This application is a division of U.S. patent application Ser. No. 16/304,064, filed on Nov. 21, 2018, which application is a U.S. National Stage Filing under 35 U.S.C. 371 from International Application No. PCT/US2017/034678, filed on May 26, 2017, and published as WO 2017/205739 on Nov. 30, 2017, which application claims the benefit of the filing date of U.S. application Ser. No. 62/341,897, filed on May 26, 2016, the disclosures of which are incorporated by reference herein.

### STATEMENT OF GOVERNMENT RIGHTS

The invention was made with the Government Support under grant 1R 21Al112803-01 by the National Institutes of Health. The Government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Nov. 11, 2024, is named 875168US2.xml and is 14, 179 bytes in size.

### BACKGROUND

Human bocavirus 1 (HBoV1) is a recently identified respiratory virus associated with acute respiratory tract infections in young children (Allander et al., 2007; Gendrel et al., 2007; Kahn, 2008; Schildgen et al., 2008; Garcia-Garcia, 2010; Don et al., 2010; Jartti et al., 2011; Brodizinski and Rhady, 2009; Martin et al., 2015) HBoV1 belongs to the Bocaparvovirus genus within the Parvoviridae (Allander et al., 2005; Cotmore et al., 2014) family. Other species in the Bocaparvovirus genus include minute virus of canines (MVC), bovine parvovirus 1 (BPV1), porcine bocavirus, and gorilla bocavirus (Cotmore et al., 2014; Sun et al., 2009; Qin et al., 2007; Cheng et al., 2010; Kapoor et al., 2010).

A unique feature of bocaparvoviruses, differed from other parvoviruses, is that they express the small nuclear phosphoprotein NP1 from an open-reading frame (ORF) located in the middle of the genome (Sun et al., 2009, Qin et al., 2007; Chen et al., 2010). NP1 is a non-structural protein and is indispensable for viral DNA replication (Sun et al., 2009; Huang et al., 2012). The role of the NP1 proteins of HBoV1 and BPV1 in viral DNA replication is conserved to that of MVC, and they are exchangeable in supporting MVC DNA replication (Sun et al., 2009). While the mechanism defining how NP1 facilitates bocaparvovirus DNA replication remains largely unknown, it has been revealed that NP1 plays an important role in processing viral precursor mRNA (pre-mRNA) to matured viral mRNA polyadenylated at the distal polyadenylation site, and therefore is important for capsid protein expression (Zan et al., 2016. Fasina et al., 2015; Sukhu et al., 2013). In addition, HBoV1 holds unique features in the genus Bocaparvovirus: it naturally replicates in non-dividing/polarized human airway epithelial cells, and the replication is dependent on the cellular DNA damage and repair machinery (Deng et al., 2016). Upon transfection, the duplex replicative form genome (RF DNA) of HBoV1 replicates in human embryonic kidney (HEK) 293 cells and produces infectious progeny virions (Huang et al., 2012). Aside from the NP1, HBoV1 expresses one large non-structural protein, NS1, and three other small nonstructural proteins, NS2, NS3, and NS4 (Shen et al., 2015). They share a C-terminus of 184 amino acids (aa). NS2 is indispensable for viral replication during infection of polarized human airway epithelial cells (Shen et al., 2015), whereas NS2-4 proteins are dispensable for viral DNA replication in HEK293 cells.

Parvovirus replication generates monomer duplex and concatemeric duplex DNA (mRF and dRF DNA, respectively) replicative intermediates via a unidirectional strand-displacement mechanism, from which the ends of progeny genomes are excised by viral replication initiator protein Rep78/68 or NS1 (Cotmore et al., 2005; Berns, 1990; Ward, 2006). For homotelomeric parvoviruses (e.g., adeno-associated virus [AAV]), in which the two genomic termini are inverted in sequence and identical in structure, the replication process is symmetrical. The tip of the T structure formed on the termini is critical for Rep78/68 binding to the replication origin (Ryan et al., 1996). For heterotelomeric autonomous parvoviruses (e.g., MVM and HBoV1), in which the two genomic termini are dissimilar, the left-end hairpin (LEH) structure that has the junction resolution site is critical to produce the ssDNA genome for progeny virion production, and the right-end hairpin (REH) structure contains the terminal resolution site (TRS) that is required to replicate viral RF DNA (Cotmore et al., 2015). In MVM, the cruciform structure at REH is required for RF DNA replication (Cotmore et al., 2000).

At either hairpin end of both homotelomeric and heterotelomeric viruses, there is a replication origin sequence that contains Rep78/68 or NS1 binding elements (RBEs/NSBEs) and TRS which is recognized and nicked by Rep78/68 or NS1 (Ryan et al., 1996; Cotmore et al., 1994, 2000, 2015). The binding and nicking properties characterized by in vivo or in vitro studies suggest that RBE or NSBE is several tetra-nucleotide repeats, which are directly recognized by the origin binding domain (OBD) of the Rep78/68 or NS1. The TRS is normally 7-17-nt (nucleotide) ahead of the RBE or NSBE at the 5′ end. The genome structure of HBoV1 is unique among these heterotelomeric parvoviruses. The LEH forms a rabbit-ear structure of 140-nt with mismatched nucleotides (“bubbles”) inside, and the REH consists of a perfect palindromic sequence of 200-nt in length (Huang et al., 2012). Of note, the REH of two other bocaparvoviruses, MVC and BPV1, are able to form a cruciform structure (Sun et al., 2009).

### SUMMARY

Because of the unique REH structure of the HBoV1 genome, the minimal requirements for terminal resolution of HBoV1 both in cis and in trans were defined using the duplex HBoV1 genome in HEK293 cells. A 46-nt minimal replication origin at the REH of HBoV1 (OriR) was identified, which contains a TRS and an unconventional NSBE. In addition, properties of nonstructural proteins NS1 and NP1 during viral DNA terminal resolution at the OriR were identified. In particular, by using the duplex replicative form of the HBoV1 genome in human embryonic kidney (HEK) 293 cells, the HBoV1 minimal replication origin (OriR) was identified at the right-end hairpin. Mutagenesis analyses confirmed the putative NS1 binding and nicking sites within the OriR. Of note, unlike the large non-structural protein (Rep78/68 or NS1) of other parvoviruses, HBoV1 NS1 did not specifically bind OriR in vitro, indicating that other viral components or cellular proteins are required for the NS1 binding to the OriR. In vivo studies demonstrated that residues responsible for NS1 binding and nicking are within the origin-binding domain. Further analysis identified that the small non-structural protein NP1 is required for terminal resolution of HBoV1 DNA at OnR. The NP1 and other viral non-structural proteins (NS1-4) colocalized within the viral DNA replication centers in both OriR-transfected cells and virus-infected cells, highlighting a direct involvement of the NP1 in the terminal resolution of viral DNA. Overall, the present study revealed characteristics of HBoV1 terminal resolution at OriR.

The disclosure provides, in one embodiment, a vector having a recombinant BoV genome useful to provide a helper-virus free (“helper-free”) preparation which genome comprises a LEH, a 3′ UTR (e.g., encompassing nucleotides 5221 to 5291 of SEQ ID NO:1), and a REH, wherein the REH includes a 46 nucleotide sequence comprising an OriR, e.g., an OriR having a TRS and NS1 nicking and binding site, and wherein the genome includes a mutation in one or more of NS2, NS3, NS4, VP1, VP2 or VP3, and or any combination thereof, and optionally a mutation in one or more of NS1 or NP1, so that NS2 protein, NS3 protein, NS4 protein, VP1 protein, VP2 protein or VP3 protein, or any combination thereof, and optionally also NS1 protein and/or NP1 protein, is/are not expressed from the vector. A mutation includes a deletion of one or more nucleotides, an insertion of one or more nucleotides, or a substitution of one or more nucleotides, e.g., a substitution that creates a stop codon, or creates or eliminates a splice site, or any combination thereof. In one embodiment, the genome comprises an expression cassette encoding a heterologous gene product. In one embodiment, the gene product encodes a therapeutic protein. In one embodiment, the gene product is a viral, bacterial, tumor, parasite, or fungal antigen. In one embodiment, the gene product is cystic fibrosis transmembrane conductance regulator, β-globin, γ-globin, tyrosine hydroxylase, glucocerebrosidase, aryl sulfatase A, factor VIII, dystrophin, alpha 1- antitrypsin, surfactant protein SP-D, SP-A or SP-C, erythropoietin, HBoV protein, influenza virus protein, RSV protein, a neutralizing antibody or an antigen binding fragment thereof, SARS virus protein, DNA endonuclease for gene editing, e.g., CRISPR associated protein 9 (Cas9), zinc finger nuclease, Transcription activator-like effector nucleases (TALEN), Argonaute, or a cytokine, e.g., IFN-alpha, IFN-gamma, TNF. IL-1, IL-17, or IL-6. In one embodiment, the BoV genome comprises a human genomic DNA segment as template for gene correction through homologous recombination between the viral genome and genomic DNA (see FIG. 12). For example, the insert includes homology arms, e.g., >100 bp, >500 bp or >1 kb, on each side of the target alterations, e g, an insert in rBoV of about 5.8 kb of genome homology (about 2.6 kb on either site of the targeted base pairs), where the arms individually have at least 95%, 96%, 97%, 98%, 99%, or 100% sequence identity to the genome target sequences. In one embodiment, 1 to 3 bp alterations may be targeted (e.g., in a gene like CFTR). Targets in CFTR include but are not limited to the deltaF508 CFTR mutation (a deletion of 3 bp) and the G551D CFTR mutation (a 1 bp mutation). In one embodiment, an insertion (or a sequence that includes a deletion) may be 2 kb leaving 1.6 kb homology arms. Vectors for gene correction do not necessarily include a heterologous promoter or poly A. In one embodiment, the human genomic DNA segment is the exons/intron sequence at the 189 kb cystic fibrosis transmembrane conductance regulator locus. In one embodiment, OriR includes nucleotides 5357 to 5402 or 5430 of SEQ ID NO:1. In one embodiment, the OriR includes (TGT)ₐ-(TGT)_{b}-(TGT)_{c}-(TGT)_{d}, where at least two of a, b, c or d is present. In one embodiment, the nicking site includes CTATATCT.

A recombinant BoV e g, rHBoV1, helper free preparation, e g., vector generation in the absence of wild-type (WT) virus contamination, is also provided. The identification of minimal oriR allows for replication of BoV, e.g., rHBoV1, genomes without generating WT virus. Moreover, having a predominantly negative strand genome may allow for higher level of specificity as certain viral proteins could be included without viral replication to boost the immune response, e.g., the helper free virus is an attenuated BoV. In addition, having a predominantly negative strand genome may allow for higher efficiency for gene editing and/or gene correction in rBoV, as the accumulation of single strand genomes could elicit DNA damage signaling and recruit cellular DNA repair and homologous recombination factors, which facilitate the gene targeting correction. Further, the mutant genomes may enhance titers of recombinant virus and functionality of the recombinant genomes, and have applications in gene editing (homologous recombination of viral genomes with the genomic DNA) since the virus only packages one strand of the genome. By removing BoV coding regions, but not at the expense of sequences for replication and/or packaging, thereby providing a mutant BoV genome, the mutant BoV genome may be modified by adding open reading frames for certain HBoV1 proteins or other microbial proteins to enhance immune response against microbial infection. Moreover, the mutant genomes may be useful in packaging systems for chimeric AAV/HBoV1 vector. Further, a mutated REH (such as inactivated the nick site) may be incorporated into a BoV helper vector, for instance, a HBoV1 helper vector. The helper might generate replicative duplex HBoV1 genomes in transfected cells, but they are defective and would not be nicked for ss genome to produce HBoV1 replication competent virus (RCV) contamination, and such replicative forms of an HBoV1 helper might be more efficient in HBoV1 VP gene expression.

A method is provided to express a heterologous gene product in mammalian cells which employs a helper-free virus preparation having a mutant BoV genome; and infecting the cells with the virus in an amount effective to express the heterologous gene product. In one embodiment, the gene product is a therapeutic gene product, a catalytic RNA, a microRNA, RNA pre-transplicing molecule (PTM-RNA), a neutralizing antibody or an antigen binding fragment thereof, a prophylactic gene product, a polypeptide or a peptide. In one embodiment, the mammalian cell is a mammalian nasal epithelial cell, tracheobronchial cell or a lung cell. In one embodiment, the REH corresponds to nucleotides 5357 to 5430, e.g., 5357 to 5402, of SEQ ID NO:1. In one embodiment, the 3′UTR corresponds to nucleotides 5221 to 5302, e.g., 5221 to 5291, of SEQ ID NO:1.

In one embodiment, a method is provided to inhibit or treat a condition associated with aberrant expression of an endogenous gene product is provided. The method contacting a mammal at risk of or having the condition, with an effective amount of a helper-free virus composition having a mutant BoV genome, wherein the mutant genome comprises a transgene encoding at least a portion of a functional gene product, the expression of which in the mammal inhibits or treats at least one symptom of the condition. In one embodiment, the trangene encodes cystic fibrosis transmembrane conductance regulator, β-globin, γ-globin, alpha- antitrypsin, tyrosine hydroxylase, glucocerebrosidase, aryl sulfatase A, factor VIII, dystrophin or erythropoietin. In one embodiment, an agent, e.g., a proteasome inhibitor, a chemotherapeutic, a lipid lowering agent, a mucolytic agent, an antibiotic or a food additive, is further administered. In one embodiment, the agent and the virus are administered to the lung, nasal epithelium, gastrointestinal tract, or blood. In one embodiment, the virus is orally or parenterally administered.

Also provided is a method to correct endogenous gene defects in mammalian cells. The method includes administering to a mammal a rBoV, e.g., rBoV, having a segment of human genomic DNA, which serves as a template for DNA repair through homologous recombination In one embodiment, the rBoV, e.g., rHBoV1, also expresses RNA-guided DNA endonuclease, e.g., CRISPR associated protein 9 (Cas9) and guide RNA, or an engineered endonuclease, such as a zinc finger nuclease or TALEN, to introduce double strand DNA breaks (DSB) or single strand nick(s) at a desired site in the chromosome for gene correction and/or gene editing. In one embodiment, the DNA correction template and the endonuclease components may be delivered in a single rBoV vector or separately delivered using two rBoV vectors, a rBoV and chimeric rAAV/BoV vector or a rBoV vector and another non-parvovirus vector including a non-viral vector. In the case of the chimeric vector, in one embodiment, the rAAV/BoV vector may be used to express the endonuclease and gRNA, while the rBoV vector may serve as the single stranded DNA template for homologous recombination. In one embodiment, the gene to be corrected is a defective human cystic fibrosis transmembrane conductance regulator gene. In one embodiment, an agent that enhances viral transduction or expression and the virus are administered to the lung, nasal epithelium, gastrointestinal tract, or blood. In one embodiment, the virus is orally or parenterally administered, for example, to the lung, nasal epithelium or blood. In one embodiment, the virus is administered to the lung, nasal epithelium or blood.

Further provided is a method to immunize a mammal. The method includes administering to a mammal a rBoV, e.g., rHBoV, having a mutant genome and encoding a prophylactic gene product in an amount effective to prevent or inhibit microbial infection or replication. In one embodiment, the gene product is an antigen of a virus or bacteria. In one embodiment, the mammalian cells or mammal are/is contacted with the virus before the cell is contacted with an agent, such as a proteosome inhibitor. In one embodiment, the cells or mammal are/is contacted with the virus and an agent, e.g., a proteasome inhibitor, concurrently. In one embodiment, the agent and the virus are administered to the lung, nasal epithelium, gastrointestinal tract, or blood. In one embodiment, the virus is orally or parenterally administered, for example, to the lung, nasal epithelium or blood. In one embodiment, the virus is administered to the lung, nasal epithelium or blood.

In one embodiment, a method to immunize a mammal is provided. The method includes administering to a mammal an isolated rBoV comprising bocavirus capsid proteins and a mutant rBoV genome, which genome also encodes a prophylactic gene product, in an amount effective to prevent or inhibit microbial infection or replication. In one embodiment, the capsid proteins are human bocavirus proteins. In one embodiment, the capsid proteins are non-human bocavirus protein. In one embodiment, the virus is administered to the lung, nasal epithelium or blood. In one embodiment, the rBoV is a human rBoV. In one embodiment, the rBoV is a non-human rBoV. In one embodiment, the rBoV is a swine, feline or canine BoV. In one embodiment, the BoV is a human rBoV. In one embodiment, the BoV is a non-human, mammalian rBoV. In one embodiment, the mutation is a deletion or a stop codon. In one embodiment, the deletion is in VP1, VP2 or VP3, or any combination. In one embodiment, OriR includes nucleotides 5357 to 5402 or 5430 of SEQ ID NO: 1. In one embodiment, the OriR includes (TGT)ₐ-(TGT)_{b}-(TGT)_{c}-(TGT)_{d}, where at least two of a, b, c or d is present. In one embodiment, the nicking site includes CTATATCT.

Also provided are methods to prepare helper-free rBoV. In one embodiment, the proviral cis plasmid contains all the minimal BoV cis-elements for replication and packaging, that is, the minimal sequence at left side (5′ terminus), including the LEH and the minimal sequence at the right side (including the right REH, as well as nucleotides 5,22 to 5,291 at the 3′ UTR. The helper plasmid provides all viral necessary proteins in trans, e.g., via a plasmid based expression system, which does not have any cis elements, so that no homologous recombination occurs between the cis and trans plasmids to generate the wild-type genome.

Other systems (not plasmid-based) for helper-free BoV, e.g., helper-free HBoV1, vector includes but is not limited to an inducible cell line, which is integrated with an defective BoV genome, such as a defective HBoV1 genome (cis-element minus), to express viral proteins for packaging and replication under an inducible promoter. In addition, bocavirus proteins for replication and packaging may be provided using one or more exogenous vectors encoding one or more, but not all, of the bocavirus proteins for replication and packaging, which is/are introduced to cells having stably integrated vectors that express one or more, but not all of the proteins, for replication and packaging, where the exogenous vectors and the stably integrated vectors together provide for all of the bocavirus proteins for replication and packaging.

In one embodiment, a method of preparing a helper virus-free preparation is provided. The method includes providing a mutant bocavirus genome genome comprising a LEH, a 3′ UTR and a REH, wherein the REH includes a 46 nucleotide sequence comprising OriR having a TRS and a NS1 nicking or binding site, wherein the mutant genome includes a mutation in one or more of NS2, NS3, NS4, VP1, VP2 or VP3, and or any combination thereof, and optionally a mutation in NS1 or NP1 or both, so that NS2 protein, NS3 protein, NS4 protein, VP1 protein, VP2 protein or VP3 protein, or any combination thereof, and optionally NS1 protein and/or NP1 protein, is/are not expressed when the mutant genome is introduced to cells. Also provided in trans are one or more of NP1, NS1, VP1, VP2 and VP3, e.g., using one or more vectors or one or more expression cassettes for NP1, NS1, VP1, VP2 and VP3. The mutant bocavirus genome and the one or more vectors or one or more expression cassettes are introduced to cells, or the mutant BoV genome is introduced to cells that express BoV proteins in trans, and helper free virus is isolated from the cells. In one embodiment, the mutant genome comprises an expression cassette encoding a heterologous gene product. In one embodiment, the gene product encodes a therapeutic protein. In one embodiment, the gene product is a viral, bacterial, tumor, parasite, or fungal antigen. In one embodiment, the gene product is cystic fibrosis transmembrane conductance regulator, β-globin, γ-globin, tyrosine hydroxylase, glucocerebrosidase, aryl sulfatase A, factor Vill, dystrophin, alpha 1- antitrypsin, surfactant protein SP-D, SP-A or SP-C, erythropoietin, HBoV protein, influenza virus protein, RSV protein, a neutralizing antibody or an antigen binding fragment thereof, SARS virus protein, or a cytokine, e.g., IFN-alpha, IFN-gamma, TNF, IL-1, IL-17, or IL-6. In one embodiment, the bocavirus is a human bocavirus. In one embodiment, what is provided are one or more vectors that express bocavirus VP1, VP2, VP3, NP1 and NS1, but optionally do not express one or more of bocavirus NS2, NS3 or NS4, and a mutant BoV genome comprising a LEH, a 3′ UTR and a REH, wherein the REH includes a 46 nucleotide sequence comprising OriR having a TRS and NS1 nicking or binding sites, wherein the mutant genome includes a mutation in one or more of NS2. NS3, NS4, VP1, VP2 or VP3, and optionally a mutation in NS1 or NP1 or both, so that NS2 protein, NS3 protein, NS4 protein, VP1 protein, VP2 protein or VP3 protein, and optionally NS1 protein and/or NP1 protein, is/are not expressed. The one or more vectors and the mutant genome are expressed in the cells and helper free virus is collected.

### BRIEF DESCRIPTION OF THE FIGURES

FIGS. 1A-C. Identification of the cis-acting sequences and trans-acting proteins required for HBoV1 replication. (A) Diagram of HBoV1 genome and mutants. HBoV1 genome is shown as a negative sense ssDNA. Sequentially from left to right, the functional elements are left-end hairpin (LEH, 3′ end), P5 promoter, NS-coding region, NP1-coding region, VP-coding region, 3′ untranslated region (3′ UTR), and the right-end hairpin (REH, 5′ end). The wild-type infectious clone is shown as duplex form DNA (dsDNA). Non-coding sequences LEH, 3′ UTR, and REH were deleted as described below. The ORF of NS, NP1, or VP was terminated as described below. Dotted lines indicate sequence deletion; vertical bars indicate nucleotide mutations. (B&C) Southern blot analysis. The wild-type infectious clone (plHBoV1) or the indicated mutants were transfected into HEK293 cells. At 2 days post transfection, low molecular weight (Hirt) DNA was extracted and applied for Southern blotting after Dpn I digestion. One hundred twenty ng of plHBoV1 digested with Dpn I at the same condition as Hirt DNA samples was used as Dpn I digestion control (Dpn I-Dig. Ctrl). RF, replicative form DNA intermediate; dRF, double RF DNA intermediate; ssDNA, single-stranded DNA. Asterisks indicate replicated DNA from the entire HBoV1 DNA-contained plasmid. M: a size marker.

FIGS. 2A-F. Identification of the minimal replication origin (OriR) at the REH. (A) Diagram of REH deletion mutants. All REH deletion mutants were constructed based on the LEH-deleted and VP1/3-knockout plasmid plHBoV1 (LEH-VP1/3-). Group | mutants had deletions at the 3′ end from nt 5,543 to 5,383. Group II mutants shared the 3′ end deletion to nt 5,430 and were further deleted at the 5′ end from nt 5,354nt to 5,384. Group III mutants shared the 5′ end deletion to nt 5,357 except for ΔREH18, which had a 5′ end at nt 5,360. The deletion at the 3′ end started from nt 5,410nt to 5,390. (B-D) Southern blot analyses. HEK293 cells were transfected with linearized mutant HBoV1 DNAs, and Hirt DNA samples were extracted at two days post transfection. Dpnl digested Hirt DNA samples were applied for Southern blotting. (E) Quantification. Three experiments were repeated for panel D, and the RF DNA was quantified in each lane The level of RF DNA from the wild-type HBoV1 was set up as 100% (Lane 1) after normalization with the main Dpn I-digested bands. N.S., not significant; *, P<0.05; **, P<0.01. (F) Minimal replication origin (OriR). The sequence of the identified OriR is shown in duplex DNA.

FIGS. 3A-B. HBoV1 OriR replicated independent of upstream 3′ UTR. (A) Diagram of λ DNA insertion mutants. Based on pHBoV1-Ori, 200 bp, 400 bp, or 1000 bp of A DNA was inserted between 3′ UTR and REH. (B) Southern blot analysis. HEK293 cells were transfected with linearized mutants. At two days post transfection, Hirt DNA samples were extracted and digested with Dpn I before they were applied for Southern blotting. plHBoV1 (NS1-) was used as a negative control. Sixty ng of plHBoV1 was digested as Dpn I digestion control.

FIG. 4. Both NS1 and NP1 are required for replication of the Ori. HEK293 cells were transfected with HBoV1-Ori or its mutants pHBoV1 (NS1-)-Ori and PHBoV1 (NP1-)-Ori. Hirt DNA samples were extracted at two days post transfection and were digested with Dpn I for Southern blotting. Sixty ng of plHBoV1 digested by DpnI was used as a digestion control.

FIGS. 5A-G. NS and NP1 co-localized with the BrdU-chased viral DNA replication centers. (A-C) Localization of NS, NP1, and BrdU-incorporated HBoV1 DNA in transfected cells. HEK 293 cells were transfected with pBB (vector backbone), plHBoV1, or pHBoV1-Ori. At two days post transfection, the cells were incorporated with BrdU before being harvested and then applied for immunofluorescence staining. Anti-NS1C with anti-BrdU (A), anti-NP1 with anti-BrdU (B), and anti-NS1C with anti-NP1 were co-stained. (D-F) Localization of NS1, NP1, and BrdU incorporated HBoV1 DNA during infection. HAE-ALI cultures were infected by HBoV1 at an MOI of 10. At 7 days post infection, cells were incorporated with BrdU and applied for immunofluorescence analysis NS, NP1, or BrdU were co-stained in different combinations. (G) Interaction of NP1 with BrdU-chased viral DNA. Transfected cells, as indicated, were pulse chased with BrdU, and were analyzed by PLA with anti-NP1 and anti-BrdU antibody. DAPI was used to stain the nucleus. Confocal images were taken at 100× under a Nikon confocal microscope.

FIGS. 6A-B. NS and NP1 did not interact directly. HEK 293 cells were transfected with pHBoV1-Ori or mock transfected. At two days post-transfection, cells were washed, lysed and pre-cleared with normal rat serum as described below. Pre-cleared lysates were immunoprecipitated with either rat IgG and rat anti-NP1 rat serum, respectively (A), or rat IgG and rat anti-NS1C serum, respectively (B).

Immunoprecipitated proteins were analyzed by Western blotting using an anti-NS1C antibody. Four μl of the pre-cleared lysates were loaded as input controls.

FIGS. 7A-C Characterization of the putative NS1 binding element in vivo. (A) Diagram of NSBE mutants. The wild-type OriR and mutated sequences of the putative NSBE are shown. The putative NSBE include (TGT)₄, and the mutants were constructed based various mutations of the “TGT” repeats. (B) Southern blot analysis. HEK293 cells were transfected with linearized wild-type Ori and NSBE mutants as indicated. Hirt DNA samples were extracted and applied for Southern blotting after Dpn I digestion. (C) Quantification. After normalization with Dpn I-digested bands, the RF DNA bands on blots were quantified. Related levels to the RF DNA from pHBoV1-Ori (lane 9) are shown with averages and standard deviations from three independent experiments.

FIGS. 8A-B. Identification of the terminal resolution site (TRS) within OriR in vivo. (A) Diagram of the TRS mutants. The dynamic TRS identified by rapid amplification of the 3′ end of nicked DNA are indicated by arrows. The sequenced results of poly-adenosine starting sites derived from transfection of the HBoV1 full-length genome (IHBoV1) and HBoV1-Ori are shown with stipled and solid arrowheads, respectively. Mutations at seven of these polyA starting sites, as shown, were performed. (B) Southern blot analysis. Linearized HBoV1 DNAs were transfected into HEK293 cells. At two days post-transfection, Hirt DNA samples were extracted, Dpn I digested, and analyzed by Southern blotting. Sixty ng of pHBoV1-Ori was digested by Dpn I as a control.

FIGS. 9A-C. Characterization of the Ori binding domain (OBD) of the NS1 in vivo. (A) Diagram of NS1 mutants. The NS1 OBD is diagrammed to scale with putative Ori binding site and endonuclease activity shown. Based on pHBoV1-Ori, OBD mutants that have NS1 amino acid mutations at the indicated positions are shown. (B) Superimposition of three NS1 OBD mutants with the wild-type NS1 OBD structure. The NS1 OBD structures of Mut K, Mut R, and Mut Endo mutants were predicted by the web server ‘I-TASSER’ based on the wild-type structure (Zhang, 2008). The predicted structures of the mutants were individually superimposed with wild-type NS1 OBD structure using the web server ‘Superpose 1.0’ (Maiti et al., 2004). The mutated amino acids in the putative DNA binding loop K and loop R and the endonuclease activity core are labeled with boxes. (C) Southern blot analysis. Linearized HBoV1 DNAs, as indicated, were transfected into HEK293 cells. At two days post-transfection, Hirt DNA samples were extracted, digested with Dpn I, and analyzed for DNA replication using Southern blotting. Dpn I digestion control was 60 ng of pHBoV1-Ori plasmid digested with Dpn I.

FIGS. 10A-D. HBoV1 NS1 alone did not specifically bind the OriR in an in vitro setting. (A) NS1 protein purification. Five μl (15 μmol) of purified HBoV1 NS1 (lane 1) and B19V NS1 (lane 2) were analyzed on SDS-PAGE, and stained with Coomassie brilliant blue. Lane 3, 2 μg of bovine serum albumin (BSA); lane 4, a protein ladder. (B) B19V NS1 specifically binds B19V Ori in vitro. γ-ATP labeled B19V Ori (lanes 1-7) or Ori-mut (lanes 8 and 9) was incubated with (lanes 2-6 and 9) or without B19V NS1 (lanes 1 and 8) in the binding buffer with 2 μg/ml Poly [d (I-C)]. Cold Ori probe at ratios of 20 times (lane 3) and 200 times (lane 4) or cold Ori-mut probe at levels of 20 times (lane 5) and 200 times (lane 6) was added for competition. Free and shifted probes are indicated. GST protein was added as a negative control (lane 7). (C) HBoV1 NS1 did not bind HBoV1 OriR in vitro. γ-ATP labeled HBoV1 OriR was incubated with (lane 2-8) or without (lane1) HBoV1 NS1 in the binding buffer with 2 μg/ml Poly [d (I-C)]. Cold OriR and OriR-mut probes at 10 times (lanes 3 and 6), 100 times (lanes 4 and 7), and 1,000 times (lanes 5 and 8) were included for competition. Free and shifted probes are indicated. (D) Non-specific HBoV1 NS1 binding to OrR. γ-ATP labeled HBoV1 Ori was incubated with (lanes 2-8) or without (lane1) HBoV1 NS1 in the absence of Poly [d (I-C)]. Cold OriR/OriR-mut probe at 50 times (lanes 3 and 6), 10 times (lanes 4 and 7), 4 times (lanes 5 and 8) was included for competition. Free and shifted probes are indicated.

FIGS. 11A-D. HBoV1 NS1 alone or with NP1 did not specifically bind OriR in a pull-down assay. (A&B) Detection of B19V NS1 and B19V Ori binding. (A) Nuclear extract (NE) prepared from OptiB19VNS1-transfected HEK 293 cells were used to incubate with Biotin-labeled B19V Ori (Bio-Ori; lanes 3-5) and B19V Ori-mut (lane 6), followed with streptavidin-conjugated beads. Additionally, some reactions were additionally incubated with B19V Ori-mut at 30 times (lane 4) and B19V Ori at 30 times (lane 5). NS1 bands pulled down by the Bio-Ori are indicated. (B) The NS1 bands pulled down by Bio-Ori were quantified. Relative levels to the NS1 in lane 4 are shown with averages and standard deviations from three independent experiments. (C&D) Detection of HBoV1 NS1 and HBoV1 OriR binding. (C) NE prepared from OptiHBoV1NS1-transfected HEK 293 cells were used to incubate with HBoV1 OriR (lane 2), and HBoV1 OriR-mut (lane 3), followed with streptavidin-conjugated beads. NS1 detected in the input is indicated. (D) NE prepared from OptiHBoV1NS1- and OptiHBoV1NP1-transfected HEK293 cells were used to incubate with HBoV1 OriR or OriR-mut, followed with streptavidin-conjugated beads. NS1 and NP1 detected in the input are indicated.

FIG. 12. Schematic of an exemplary use of a rBoV vector for homologous recombination.

FIG. 13A-E. Exemplary BoV nucleotide sequence (SEQ ID NO: 1).

### DETAILED DESCRIPTION

### Definitions

An “origin of replication” (“ori”) is a DNA sequence, e.g., in a bocavirus, that when present in a cell may be capable of maintaining linked sequences, and/or is a site at or near where DNA synthesis initiates. As described hereinbelow an ori for bocavirus includes TRS and NS1 nicking and binding sequences (NSBEs).

A “BoV vector” as used herein refers to a BoV vector which may comprise a polynucleotide sequence not of BoV origin (i.e., a polynucleotide heterologous to AAV), typically a sequence of interest for the genetic transformation of a cell. Vector constructs, the heterologous polynucleotide is flanked by LEH and 3′UTR-REH, or a minimal OriR. The term BoV vector encompasses both BoV vector particles and BoV vector plasmids.

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest for gene therapy. Vectors include, for example, viral vectors (such as adenoviruses, parvoviruses include bocavirus and adeno-associated viruses (AAV), lentiviruses, herpesvirus and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Large varieties of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene includes at least a portion of an open reading frame of a gene which is partly or entirely heterologous (i.e., foreign) to the transgenic organism, or may represent an open reading frame or a portion thereof of a gene homologous to an endogenous gene of the organism, which portion optionally encodes a polypeptide with substantially the same activity as the corresponding full length polypeptide, e.g., wild-type polypeptide, or at least one activity of the corresponding full length polypeptide.

By “transgenic cell” is meant a cell containing a transgene. For example, a stem cell transformed with a vector containing an expression cassette can be used to produce a population of cells having altered phenotypic characteristics. A “recombinant cell” is one which has been genetically modified, e.g., by insertion, deletion or replacement of sequences in a nonrecombinant cell by genetic engineering.

The term “wild-type” or “native” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector and preferably via a replication-defective viral vector.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “enhancer” is meant a nucleic acid sequence that, when positioned proximate to a promoter, confers increased transcription activity relative to the transcription activity resulting from the promoter in the absence of the enhancer domain

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide or virus refers to a nucleic acid sequence, peptide, polypeptide or virus that is identified and separated from at least one contaminant nucleic acid, polypeptide or other biological component with which it is ordinarily associated in its natural source. Isolated nucleic acid, peptide, polypeptide or virus is present in a form or setting that is different from that in which it is found in nature. For example, a given DNA sequence (e.g., a gene) is found on the host cell chromosome in proximity to neighboring genes: RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture.

The term “recombinant DNA molecule” as used herein refers to a DNA molecule that is comprised of segments of DNA joined together by means of molecular biological techniques.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

The term “prophylactically effective amount” is meant to refer to the amount necessary to, in the case of infectious agents, prevent an individual from developing an infection, and in the case of diseases, prevent an individual from developing a disease.

The term “therapeutically effective amount” is meant to refer to the amount necessary to, in the case of infectious agents, reduce the level of infection in an infected individual in order to reduce symptoms or eliminate the infection, and in the case of diseases, to reduce symptoms or cure the individual.

“Inducing an immune response against an immunogen” is meant to refer to induction of an immune response in a naïve individual and induction of an immune response in an individual previously exposed to an immunogen wherein the immune response against the immunogen is enhanced.

“Gene expression” or “expression” refers to the process of gene transcription, translation, and post-translational modification.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

A “chimeric virus” or “chimeric viral particle” refers to a viral particle composed of at least one capsid protein and an encapsidated polynucleotide which is from a different virus.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” or “TRS,” as used herein, refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide). Similarly, a TRS or promoter that is removed from its native coding sequence and operably linked to a different coding sequence is a heterologous TRS or promoter.

“Packaging” as used herein refers to a series of subcellular events that results in the assembly and encapsidation of a viral vector. Thus, when a suitable vector is introduced into a packaging cell line under appropriate conditions, it can be assembled into a viral particle. Functions associated with packaging of viral vectors are described herein and in the art.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art, and illustrative uses of such sequences within the context of the present invention are provided below.

“Host cells,” “cell lines,” “cell cultures,” “packaging cell line” and other such terms denote higher eukaryotic cells, e g, mammalian cells, such human cells, useful in the present invention These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

A “therapeutic gene,” “prophylactic gene,” “target polynucleotide,” “transgene,” “gene of interest” and the like generally refer to a gene or genes to be transferred using a vector. Typically, in the context of the present invention, such genes are located within the rBoV vector (which vector is flanked by LEH, 3′UTR and REH and thus can be replicated and encapsidated into rBoV particles). Target polynucleotides can be used in this invention to generate rBoV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; and (vi) polynucleotides for cancer therapy, such as E1A tumor suppressor genes or p53 tumor suppressor genes for the treatment of various cancers. To effect expression of the transgene in a recipient host cell, it is operably linked to a promoter, either its own or a heterologous promoter. A large number of suitable promoters are known in the art, the choice of which depends on the desired level of expression of the target polynucleotide; whether one wants constitutive expression, inducible expression, cell-specific or tissue-specific expression, etc. The rBoV vector may also contain a selectable marker.

A “gene” refers to a polynucleotide containing at least one open reading frame that in one embodiment is capable of encoding a particular protein after being transcribed and translated.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

“Genetic alteration” refers to a process wherein a genetic element is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. The genetic element may be introduced into a chromosome or mini-chromosome in the cell; but any alteration that changes the phenotype and/or genotype of the cell and its progeny is included in this term.

“Homologous recombination” is a type of genetic recombination in which nucleotides are exchanged between two similar or identical nucleotide sequences of DNA.

“Gene targeting” is a genetic technique that uses homologous recombination to change an endogenous gene, e.g., delete the gene, remove exons, add a gene or alter the splice donor and receptor sequence, add a gene, or change one or more bases in the DNA. See exemplary disorders and associated mutations below.

| Disorder | Mutation | Chromosome |
|---|---|---|
| 22q11.2 deletion syndrome | D | 22q |
| Angelman syndrome | DCP | 15 |
| Canavan disease | | 17p |
| Charcot-Marie-Tooth disease | | |
| Color blindness | P | X |
| Cri du chat | D | 5 |
| Cystic fibrosis | P | 7q |
| Down syndrome | C | 21 |
| Duchenne muscular dystrophy | D | Xp |
| Haemochromatosis | P | 6 |
| Haemophilia | P | X |
| Klinefelter syndrome | C | X |
| Neurofibromatosis | | 17q/22q/? |
| Phenylketonuria | P | 12q |
| Polycystic kidney disease | P | 16 (PKD1) or 4 (PKD2) |
| Prader-Willi syndrome | DC | 15 |
| Sickle-cell disease | P | 11p |
| Spinal muscular atrophy | DP | 5q |
| Tay-Sachs disease | P | 15 |
| Turner syndrome | C | X |

P—Point mutation, or any insertion/deletion entirely inside one gene;
D—Deletion of a gene or genes;
C—Whole chromosome extra, missing, or both (chromosome abnormality);
T—Trinucleotide repeat disorders: gene is extended in length

“Gene editing” is a type of genetic engineering in which DNA is inserted, deleted or replaced in the genome of an organism using a recombinant nuclease, e.g., a heterologous nuclease.

“Gene correction template” is the exogenous template that is used to introduce DNA into, delete DNA from or otherwise replace or alter DNA in the genome of an organism.

A cell is said to be “stably” altered, transduced or transformed with a genetic sequence if the sequence is available to perform its function during extended culture of the cell in vitro. In some examples, such a cell is “inheritably” altered in that a genetic alteration is introduced which is also inheritable by progeny of the altered cell.

The terms “polypeptide” and “protein” are used interchangeably herein to refer to polymers of amino acids of any length The terms also encompass an amino acid polymer that has been modified: for example, disulfide bond formation, glycosylation, acetylation, phosphonylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof, that retains the desired biochemical function of the intact protein. Similarly, references to CFTR, and other such genes for use in gene therapy (typically referred to as “transgenes” to be delivered to a recipient cell), include polynucleotides encoding the intact polypeptide or any fragment or genetically engineered derivative possessing the desired biochemical function.

A preparation of virus is said to be “substantially free” of helper virus if the ratio of infectious virus particles to infectious helper virus particles is at least about 102:1; e.g., at least about 104:1, including at least about 106:1 or at least about 108:1. Preparations may also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the capsid proteins VP1, VP2 and VP3). “Efficiency” when used in describing viral production, replication or packaging refers to useful properties of the method: in particular, the growth rate and the number of virus particles produced per cell. “High efficiency” production indicates production of at least 100 viral particles per cell; e.g., at least about 10,000 or at least about 100,000 particles per cell, over the course of the culture period specified.

An “individual” or “subject” treated in accordance with this invention refers to vertebrates, particularly members of a mammalian species, and includes but is not limited to domestic animals, sports animals, and primates, including humans.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency, infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent.

The practice of the present invention will employ, unless otherwise indicated, conventional techniques of molecular biology, virology, microbiology, recombinant DNA, and immunology, which are within the skill of the art. Such techniques are explained fully in the literature. See, e.g., Sambrook et al., 1989; Gait, 1984; Freshney, 1987; the series Methods in Enzymology (Academic Press, Inc.); Miller et al., 1987; Weir et al., 1996; Ausubel et al., 1998; Coligan et al., 1991; Coligan et al., 1995; and Scopes 1994.

rBoV vectors

Besides prophylactic or therapeutic gene products, recombinant BoV vectors and/or viruses can also comprise polynucleotides that do not encode proteins, including, e.g., polynucleotides encoding for antisense mRNA (the complement of mRNA) which can be used to block the translation of normal mRNA by forming a duplex with it, and polynucleotides that encode ribozymes (RNA catalysts). In addition selected pairs of rBoV vectors having portions of open reading frames flanked by appropriately placed splice acceptor sites and/or splice donor sites, or having transcription regulatory sequences such as a heterologous enhancer, a heterologous promoter, or a heterologous enhancer and a promoter, may be employed.

A BoV vector of the invention may comprise a polynucleotide that is heterologous to BoV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the BoV genomic coding region, but is generally flanked on either side by LEH, 3′UTR and REH.

A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rHBoV vector. A number of helper-virus-inducible promoters have also been described, including the adenovirus early gene promoter which is inducible by adenovirus E1A protein, the adenovirus major late promoter; the herpesvirus promoter which is inducible by herpesvirus proteins such as VP16 or 1CP4, as well as vaccinia or poxvirus inducible promoters.

Removal of one or more BoV genes is in any case desirable, to reduce the likelihood of generating replication-competent virus (“RCV”). Accordingly, sequences encoding structural and/or non-structural, or both, may be removed, since the functions provided by these genes can be provided in trans

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by BoV LEH, 3′UTR and/or REH and in one embodiment do not share any substantial homology with the BoV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCV.

The BoV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the BoV vector and complementary packaging gene(s), if any, are provided in the form of bacterial plasmids, BoV particles, or any combination thereof. In other embodiments, either the BoV vector sequence, the packaging gene(s), or both, are provided in the form of genetically altered (e.g., inheritably altered) eukaryotic cells. The development of host cells inheritably altered to express the BoV vector sequence, BoV packaging genes, or both, provides an established source of the material that is expressed at a reliable level.

A variety of different genetically altered cells can thus be used in the context of this invention. By way of illustration, a mammalian host cell may be used with at least one intact copy of a stably integrated BoV vector A BoV packaging plasmid can be used to supply replication functions. Alternatively, a stable mammalian cell line can be used to supply replication functions. The BoV VP genes, providing the encapsidation proteins, can be provided together with a BoV NS1 and/or NP1 gene or separately. Other combinations are possible and included within the scope of this invention.

Uses of rBoV

The rBoV can be used for administration to an individual for purposes of gene therapy or vaccination. Suitable diseases for therapy include but are not limited to those induced by viral, bacterial, or parasitic infections, various malignancies and hyperproliferative conditions, autoimmune conditions, and congenital deficiencies.

Gene therapy can be conducted to enhance the level of expression of a particular protein either within or secreted by the cell. Gene therapy can also be conducted to edit genomic DNA through homologous recombination with the viral genome (e.g., correct a gene defect encoded in the endogenous gene loci). Vectors of this invention may be used to genetically alter cells either for gene marking, replacement of a missing or defective gene, or insertion of a therapeutic gene. Alternatively, a polynucleotide may be provided to the cell that decreases the level of expression. This may be used for the suppression of an undesirable phenotype, such as the product of a gene amplified or overexpressed during the course of a malignancy, or a gene introduced or overexpressed during the course of a microbial infection. Expression levels may be decreased by supplying a therapeutic or prophylactic polynucleotide comprising a sequence capable, for example, of forming a stable hybrid with either the target gene or RNA transcript (antisense therapy), capable of acting as a ribozyme to cleave the relevant mRNA or capable of acting as a decoy for a product of the target gene. Vectors of this invention may also be used to deliver gene-editing components and a single stranded DNA template to genetically correct gene defects through homologous recombination. For example, negative single strand rBoV genomes without positive strands may facilitate homologous combination, e.g., in conjunction with a DNA endonuclease that cleaves at the target site of homology in the genome that is contained within the rBoV genome. In this case, the rBoV genome may encode, for example, CRISPR/Cas9 and a guide RNA (gRNA) and the site of genomic homology targeted for gene editing. Conversion of a subset of the rBoV genomes allows for expression of the Cas9/gRNA while other rBoV genomes may remain single stranded. An advantage of rBoV over rAAV is that the rAAV vector system contains equal percentages of positive and negative strand genomes while the rBoV vector system contains only the negative strand, which can anneal after infection and reduce the efficiency of HR. In another example, a rBoV genome may be employed along with a guide DNA (gDNA) for use with the NgAgo-gDNA gene editing system (see, Gao et al., Nature Biotech, 2016, doi: 10.1038/nbt.3546, the disclosure of which is incorporated by reference herein). In this system, a region of the rBoV genome contains an expression cassette for the Argonaute (NgAgo), and the gDNA (5′ phosphorylated single strand guide DNA) is introduced using different delivery methods including non-viral delivery system. Conversion of a subset of rBoV genome allows for expression of the NgAgo while other rBoV genomes will remain single stranded and serves as gene correction template.

The introduction of the rHBoV vectors by the methods of the present invention may involve use of any number of delivery techniques (both surgical and non-surgical) which are available and well known in the art. Such delivery techniques, for example, include vascular catheterization, cannulization, injection, inhalation, endotracheal, subcutaneous, inunction, topical, oral, percutaneous, intra-arterial, intravenous, and/or intraperitoneal administrations. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

In particular, for delivery of a vector of the invention to a tissue, any physical or biological method that will introduce the vector to a host animal can be employed. Vector means both a bare recombinant vector and vector DNA packaged into viral coat proteins, as is well known for administration. Simply dissolving a chimeric or THBoV vector in phosphate buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). Pharmaceutical compositions can be prepared as injectable formulations or as topical formulations to be delivered to the muscles by transdermal transport Numerous formulations for both intramuscular injection and transdermal transport have been previously developed and can be used in the practice of the invention. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the chimeric or rHBoV vector as a free acid (DNA contains acidic phosphate groups) or a pharmacologically acceptable salt can be prepared in water suitably mixed with a surfactant such as hydroxypropylcellulose. A dispersion of viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the chimeric or rHBoV vector in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the methods of preparation include but are not limited to vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

Of interest is the correction of the genetic defect of cystic fibrosis, by supplying a properly functioning cystic fibrosis transmembrane conductance regulator (CFTR) to the airway epithelium. Thus, the use of chimeric or rHBoV vectors encoding native CFTR protein, and mutants and fragments thereof, is envisioned.

Compositions of this invention may be used in vivo as well as ex vivo. In vivo gene therapy comprises administering the vectors of this invention directly to a subject. Pharmaceutical compositions can be supplied as liquid solutions or suspensions, as emulsions, or as solid forms suitable for dissolution or suspension in liquid prior to use. For administration into the respiratory tract, one mode of administration is by aerosol, using a composition that provides either a solid or liquid aerosol when used with an appropriate aerosolubilizer device. Another mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

An effective amount of virus is administered, depending on the objectives of treatment. An effective amount may be given in single or divided doses. Where a low percentage of transduction can cure a genetic deficiency, then the objective of treatment is generally to meet or exceed this level of transduction. In some instances, this level of transduction can be achieved by transduction of only about 1 to 5% of the target cells, but is more typically 20% of the cells of the desired tissue type, usually at least about 50%, at least about 80%, at least about 95%, or at least about 99% of the cells of the desired tissue type. As a guide, the number of vector particles present in a single dose given by bronchoscopy will generally be at least about 1×10¹², e.g., about 1×10^{13, 1×10}¹⁴, 1×10¹⁵ or 1×10¹⁶ particles, including both DNAse-resistant and DNAse-susceptible particles. In terms of DNAse-resistant particles, the dose will generally be between 1×10¹² and 1×10¹⁶ particles, more generally between about 1×10¹² and 1 × 10¹⁵ particles. The treatment can be repeated as often as every two or three weeks, as required, although treatment once in 180 days may be sufficient.

To confirm the presence of the desired DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence of a polypeptide expressed from a gene present in the vector, e.g., by immunological means (immunoprecipitations, immunoaffinity columns, ELISAs and Western blots) or by any other assay useful to identify the presence and/or expression of a particular nucleic acid molecule falling within the scope of the invention.

To detect and quantitate RNA produced from introduced DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the DNA segment in question, they do not provide information as to whether the DNA segment is being expressed. Expression may be evaluated by specifically identifying the polypeptide products of the introduced DNA sequences or evaluating the phenotypic changes brought about by the expression of the introduced DNA segment in the host cell.

Thus, the effectiveness of the genetic alteration can be monitored by several criteria, including analysis of physiological fluid samples, e.g., urine, plasma, serum, blood, cerebrospinal fluid or nasal or lung washes. Samples removed by biopsy or surgical excision may be analyzed by in situ hybridization, PCR amplification using vector-specific probes, RNAse protection, immunohistology, or immunofluorescent cell counting. When the vector is administered by bronchoscopy, lung function tests may be performed, and bronchial lavage may be assessed for the presence of inflammatory cytokines.

The treated subject may also be monitored for clinical features, and to determine whether the cells express the function intended to be conveyed by the therapeutic or prophylactic polynucleotide.

The decision of whether to use in vivo or ex vivo therapy, and the selection of a particular composition, dose, and route of administration will depend on a number of different factors, including but not limited to features of the condition and the subject being treated. The assessment of such features and the design of an appropriate therapeutic or prophylactic regimen is ultimately the responsibility of the prescribing physician.

The foregoing description provides, inter alia, methods for generating high titer preparations of recombinant chimeric viruses or rHBoV that are substantially free of helper virus (e.g., adenovirus) and cellular proteins. It is understood that variations may be applied to these methods by those of skill in this art without departing from the spirit of this invention.

The invention will be described the following non-limiting example.

### Example

Parvoviruses are single-stranded DNA viruses that use the palindromic structures at the ends of the viral genome for their replication. The mechanism of parvovirus replication has been studied mostly in Dependoparvovirus adeno-associated virus 2 (AAV2) and Protoparvovirus minute virus of mice (MVM). Human bocavirus 1 (HBoV1), a Bocaparvovirus, HBoV1 was first identified in 2005. HBoV1 causes acute respiratory tract infections in young children. HBoV1 infects and replicates autonomously only in well-differentiated human airway epithelial cells. However, the duplex HBoV1 genome replicates in HEK293 cells, and produces progeny virions that are infectious in well-differentiated airway epithelial cells.

As described below, both cis-acting elements and trans-acting proteins that are required for HBoV1 DNA terminal resolution in HEK293 cells were identified. The minimal replication origin was localized to a 46-nt sequence in the right-end hairpin, which contains both NS1 nicking and binding sites. The identification of these elements of HBoV1 DNA replication acting both in cis and in trans allows for antiviral strategies targeting terminal resolution, and for design of next generation recombinant HBoV1 vectors, a tool for gene therapy of lung diseases.

### Materials and Methods

Cell culture

HEK293 cells (CRL-1573, ATCC) were cultured in Dulbecco's Modified Eagle Medium (DMEM) with 10% fetal calf serum (FCS). Cells were incubated at 37° C. with 5% CO₂. HEK 293F cells (Life Technologies/Thermo Fisher Scientific Inc, Carlsbad, CA) were cultured in suspension in Freestyle™ 293 medium (Life Technologies) at 37° C. with 8% CO₂.

Primary human airway epithelium cultured at an air-liquid interface (HAE-ALI) was generated and cultured as previously described (Huang et al., 2012: Deng et al., 2016). HAE-ALI cultures that had a transepithelial electrical resistance (TEER) of >1000 Ω·cm² were selected to use in this study.

### Plasmid DNA Construction

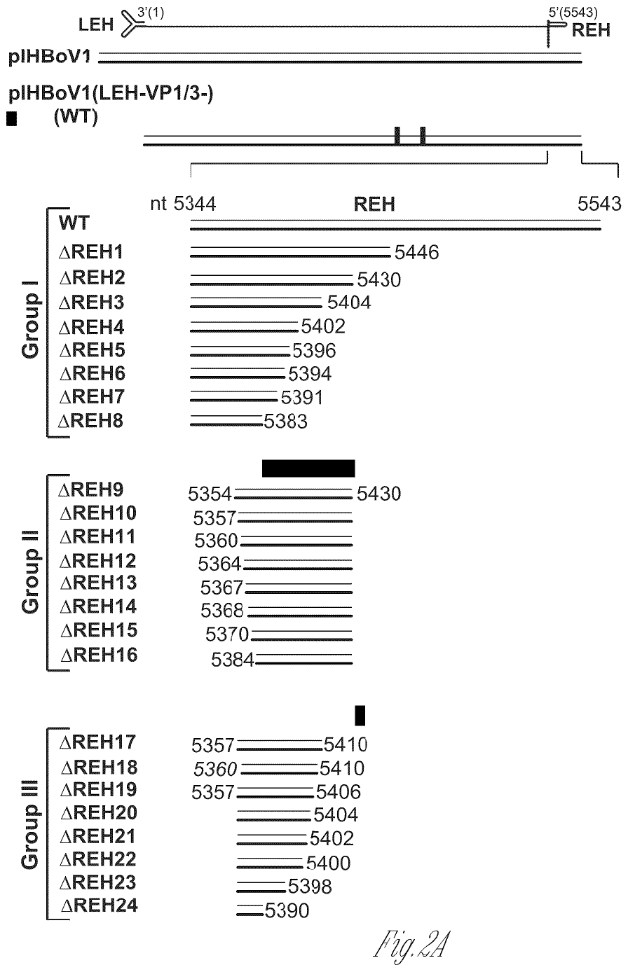

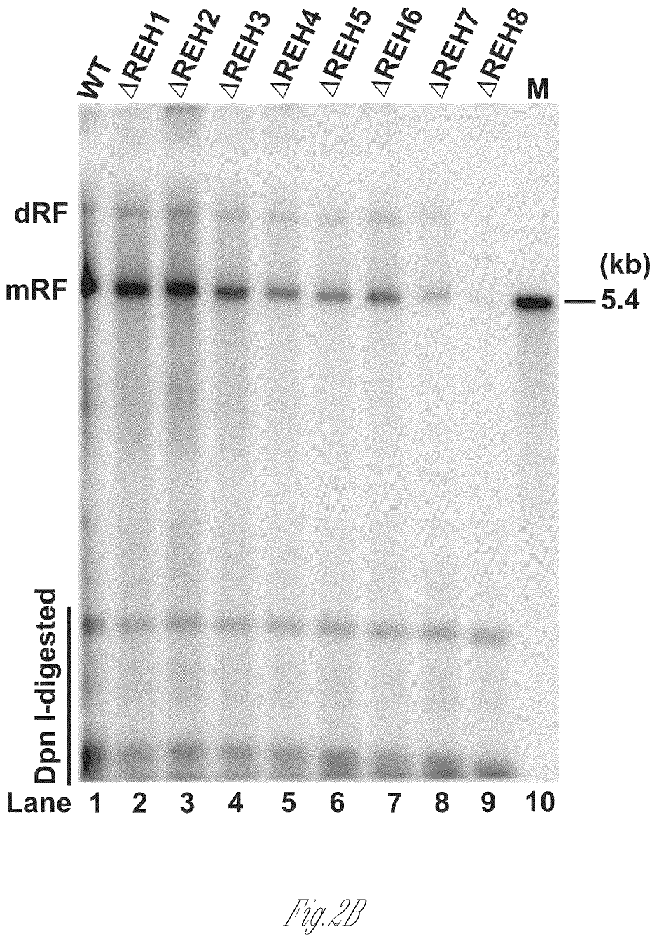

(i) plHBoV1-based constructs: The parent HBoV1 infectious clone (plHBoV1) has been described previously (Huang et al., 2012). The LEH-deleted plasmid plHBoV1 (LEH-) was constructed by deleting the HBoV1 genome sequence of nt 1-140 in plHBoV1. plHBoV1 (NS1-) and plHBoV1 (NP1-), which have the NS1 and NP1 ORFs, respectively, early terminated, and plHBoV1 (VP1/3-) that has both the capsid proteins VP1 and VP3 ORFs early terminated, were previously described (Huang et al., 2012). plHBoV1 (REH-), the REH-deleted plasmid, was constructed by deleting the HBoV1 sequence of nt 5,303-5,543 in plHBoV1. plHBoV1 (LEH-VP1/3-) was constructed by deleting LEH and early terminating VP1 and VP3 ORFs Based on the plHBoV1 (LEH-VP1/3-), an Xba I was inserted at nt 5,344 to construct REH-truncated mutants pΔREH1-24, which are diagrammed in FIG. 2. pHBoV1 (3′UTR-), the 3′ untranslated region (3′ UTR)-deleted plasmid, was constructed by deleting the HBoV1 sequence of nt 5,221-5,291 in plHBoV1. plHBoV1 (VP-) was constructed by deleting the HBoV1 sequence of nt 3,380-5,119 in plHBoV1.

(ii) pHBoV1-Ori and pHBoV1-Ori-based constructs: pΔREH21 was renamed pHBoV1-Ori that has the minimal replication origin at the REH (OriR) of nt 5,357-5,402. pHBoV1X200Ori, pHBoV1/400Ori, and pHBoV1/1000On were constructed by inserting Lambda (λ) DNA sequences of 200 bp (nt 10,030-10,230), 400 bp (nt 10,030-10,430), and 1,000 bp (nt 10,030-11,030), respectively, in front of the Ori in pHBoV1Ori.

pHBoV1 (NS1-)-Ori and pHBoV1 (NP1-)-Ori were constructed by early terminating the NS1 and NP1 ORFs, respectively, in pHBoV1-Ori using strategies described previously in Huang et al. (2012).

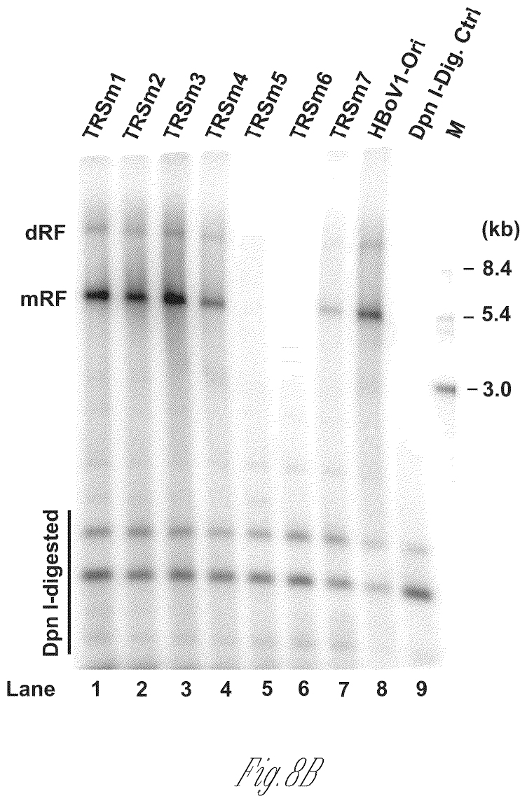

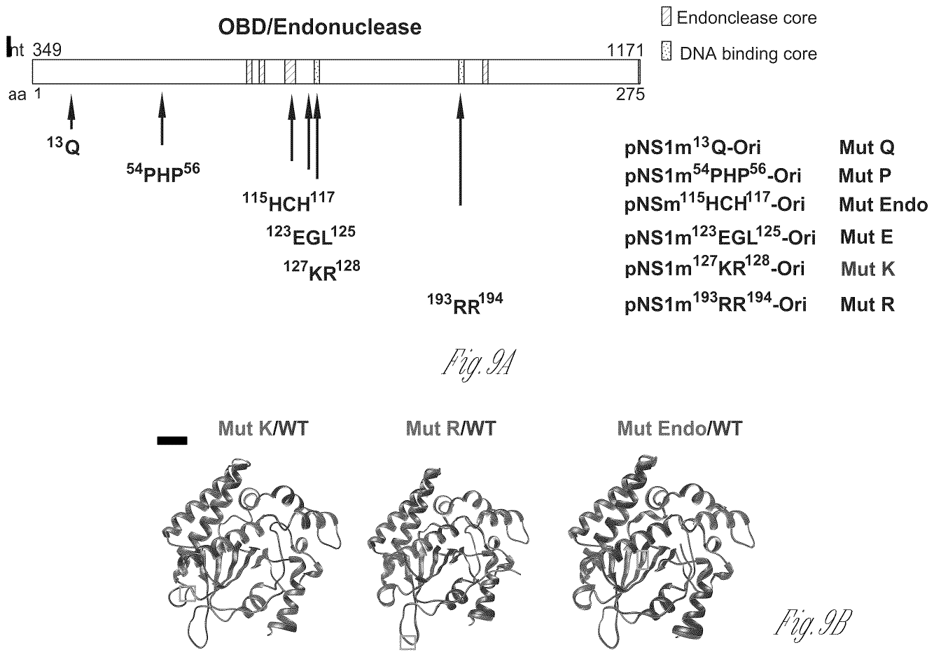

pNS1m¹³Q-Ori (Mut Q), pNS1m⁵⁴PHP⁵⁶-Ori (Mut P), pNS1m¹²³EGL¹²⁵-Ori (Mut E), pNS1m¹¹⁵HCH¹¹⁷-Ori (Mut Endo), pNS1m¹²⁷KR¹²⁸-Ori (Mut K), and pNS1m¹⁹³RR¹⁹⁴-Ori (Mut R) were constructed by mutating the amino acids as indicated by the positions to alanine(s) in the context of pHBoV1-Ori. They are diagrammed in FIG. 9.

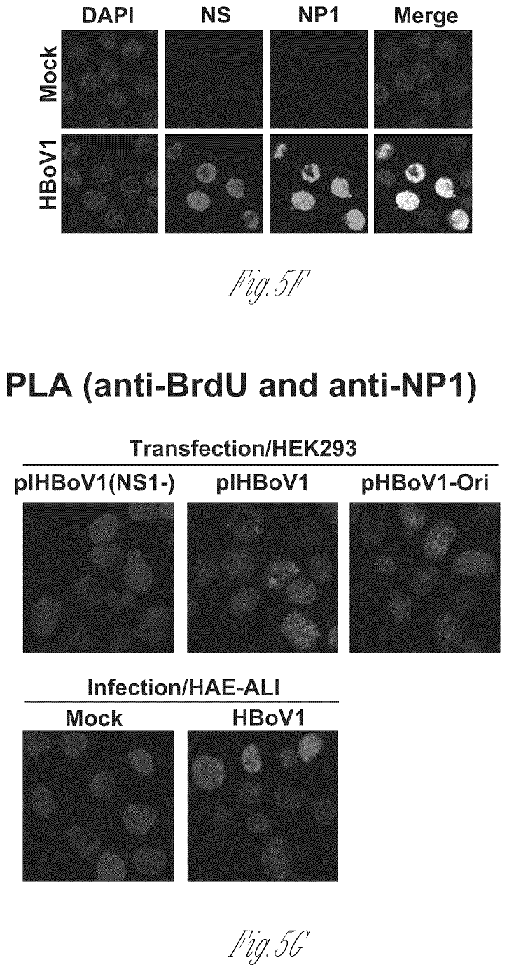

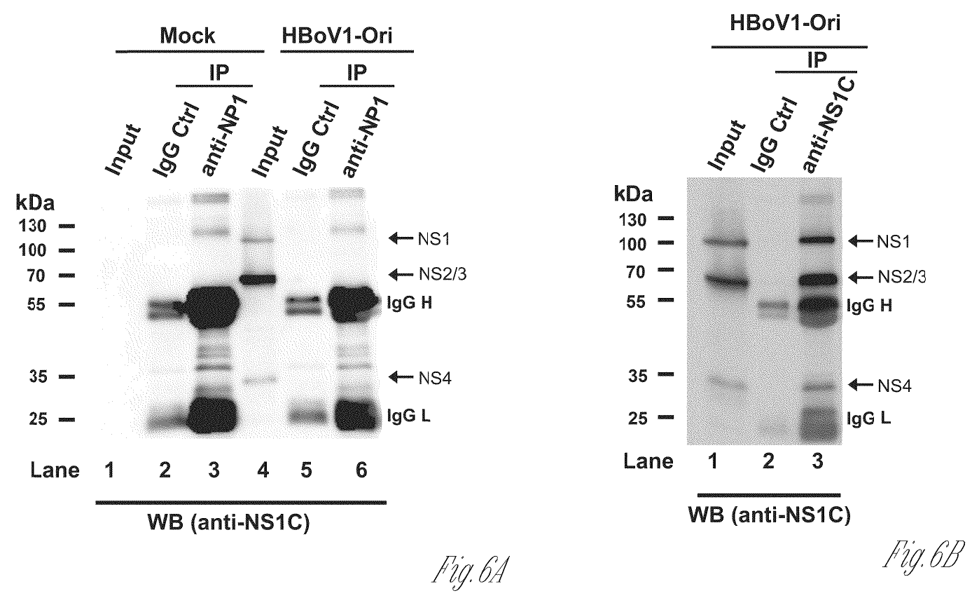

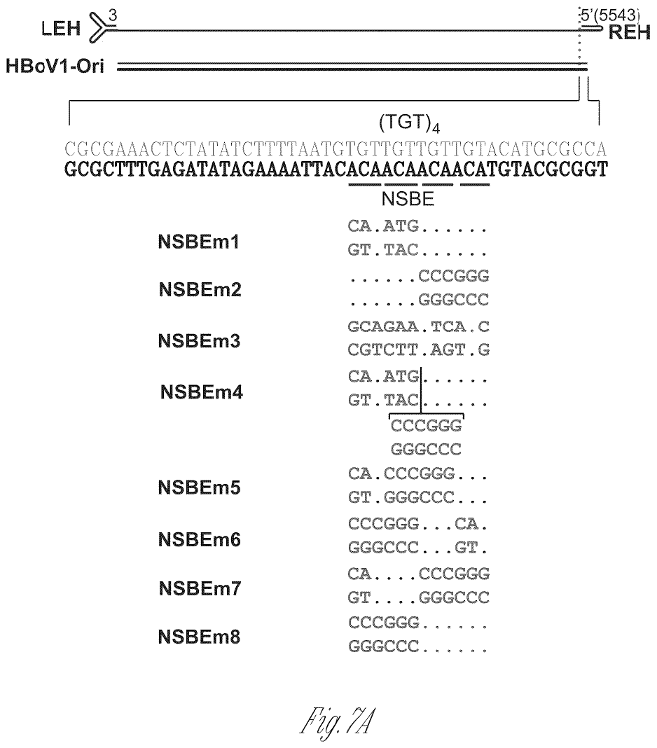

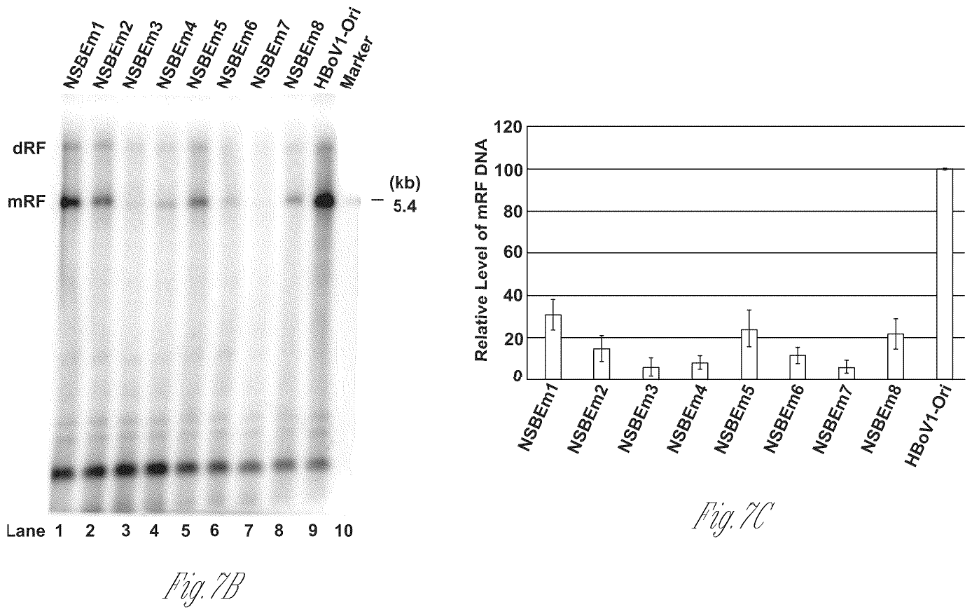

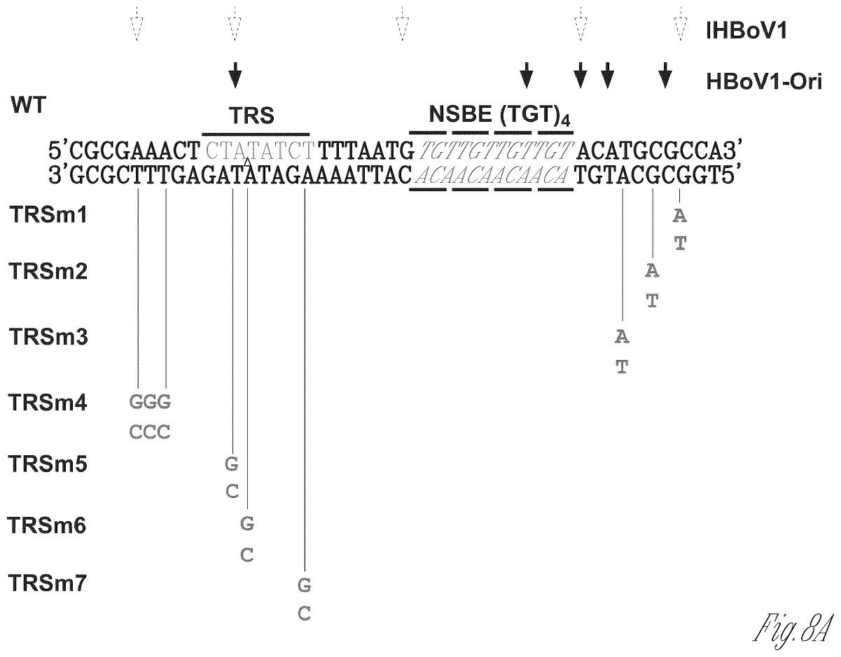

pNSBEm1-8 and pTRSm1-7 plasmids were constructed by introducing various mutations in the putative NSBEs and TRS of the OriR, respectively, in pHBoV1-Ori. These plasmids are diagrammed in FIG. 7A and FIG. 8A, respectively, with mutations shown.

(iii) HBoV1 NS1 and B19V NS1-expressing plasmids: HBoV1 and B19V NS1 coding sequences were optimized for mammalian cell expression at Integrated DNA Technologies, Inc. (IDT, Coralville, IA), tagged with a Flag tag at the C-terminus, and were cloned in pCI expression vector (Promega, Madison, WI), resulting in pOptHBoV1NS1 and pOptB19VNS1.

All nucleotide numbers of HBoV1 and Lambda (A) DNA are referred to as Genbank accession no. JQ923422 and NC_001416, respectively, unless otherwise specified.

### In Vivo DNA Replication Analysis

HEK293 cells were seeded in 6 well plates or 60 mm dishes one day before transfection. At a confluence of 70%, cells were transfected with LipoD293 reagent (SignaGen, Gaithersburg, MD) or Lipofactamine and Plus reagent (Life Technologies) following the manufacturers' instructions.

Low-molecular weight DNA (Hirt DNA) was extracted and digested with Dpn I, followed by Southern blotting. These steps were performed exactly as described in Guan et al. (2009). After hybridization, the membrane was exposed to a phosphor screen. The screen was then scanned on a phosphor imager (GE Typhoon FLA 9000, Fuji). The developed image was processed and analyzed using ImageQuant TL8.1 software (GE Healthcare, Marlborough, MA).

### BrdU Incorporation, Immunofluorescence (IF) Assay, and Proximity Ligation Assay (PLA)

For virus-infected differentiated airway epithelial cells, we treated the infected HAE-ALI treated with 5 mM EDTA for 5 minutes and then trypsinized the infected cells off the insert. We resuspended about 1×10⁵cells in 1 ml of the ALI medium with BrdU (Sigma, St Louis, MO) at 30 UM for 20 minutes. For transfected HEK293 cells, at two days post-transfection, the cells were incubated with BrdU for 30 minutes, followed a previously published method (Luo et al., 2013). Then, the labeled cells were cytospun onto slides for IF analysis.

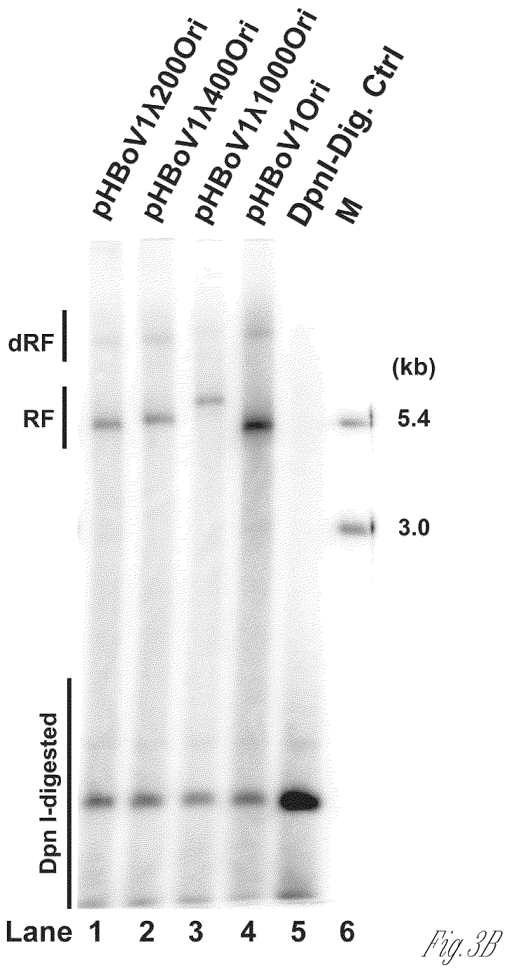

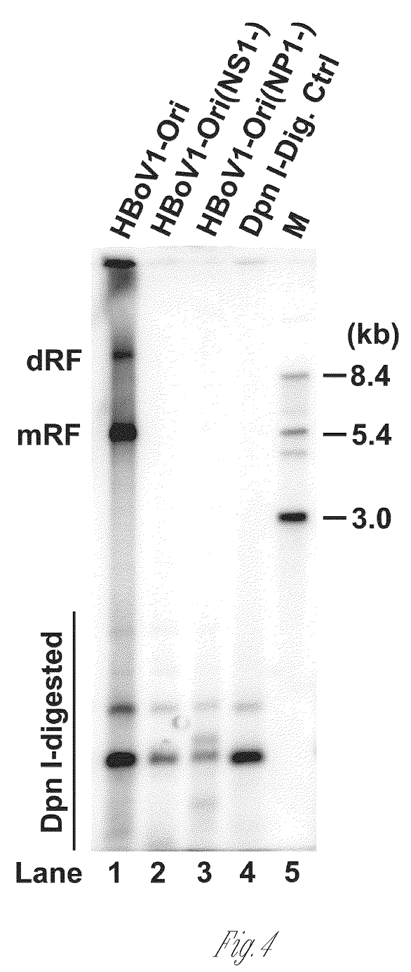

IF analysis was performed following a method described in Luo et al. (2013) and Luo et al (2011), with antibodies against proteins or BrdU as indicated in FIG. 5. Confocal images were taken with an Eclipse C1 Plus confocal microscope (Nikon) controlled by Nikon EZ-C1 software. DAPI (4′0.6-diamidino-2-phenylindole) was used to stain the nucleus.

The Duolink PLA Kit (Sigma) was used to perform PLA according to the manufacturer's instructions as previously described in Deng et al. (2016).

### Antibody Production and Antibodies Used

HBoV1 anti-NS1C antibody was produced as previously described (Chen et al., 2010), which reacts with all four NS proteins (NS1-4) (Shen et al., 2015). All animal procedures were approved by the Institutional Animal Care and Use Committee of the University of Kansas Medical Center. Rabbit anti-HBoV1 NP1 antibody (Mihaylov et al., 2014) was a kind gift from Dr Peter Tattersall at Yale University.

Rabbit anti-BrdU (cat no: 600-401-C29, Rockland, Limerick, PA), mouse anti-Flag monoclonal antibody (Sigma, St. Louis, MO), and secondary antibodies for immunofluorescence assay (Jackson ImmunoResearch Inc., West Grove, PA) were purchased.

### Rapid Amplification of the 3′ End of the Nicked DNA

DNA tailing reaction was performed using terminal transferase (NEB, Ipswich, MA). Basically, the reaction was composed of 5.0 μL of 2.5 mM CoCl₂, 1 μL of 10-50 times diluted Hirt DNA (from about 500 cells), 0.5 μL of 10 mM dATP, 0.5 μL of Terminal Transferase (20 units/uL), and deionized H₂O in a final volume of 50 μL. The reaction was incubated at 37° C. for 30 minutes. Two μL of the product was used as template for PCR amplification using forward primer (5′-CTG TCT AGA ATG ATC AAT GTA TGC CAG-3′ (SEQ ID NO: 2), nt 5,121 to nt 5, 138) and reverse primer (5′-is CAC GGA TCC TTT TTT TTT TTT TTT T-3′ (SEQ ID NO:3). Amplified fragments were cloned into pcDNA3 (Life Technologies) through Xba I and BamH I sites. Twenty positive clones were sequenced.

### Protein Expression and Purification

One 500 mL suspension culture of 293F cells (10⁶ cells/mL) was transfected with pOpti-HBoVNS1 or pOptiB19VNS1, using TransIT-PRO® following the manufacturer's instructions. At 3 days post-transfection, cells were lysed in L buffer (50 mM Tris pH 7.4, 150 mM NaCl, 1 mM EDTA, 1% Triton-X100, 1 mM dithiothreitol (DTT), 5 mM ATP, and 5 mM MgCl₂) supplemented with protease inhibitors (Cat. No. S8820, Sigma). Crude lysate was sonicated for 3 minutes at a frequency of 15 seconds/25 seconds on/off, puise 70% by the VCX130 sonicator (Sonics & Materials Inc., Newtown, CT). The lysate was then centrifuged at 10,000 rpm for 15 minutes and filtrated through a 0.2 μm filter. The filtered lysate was incubated with 1 mL of PBS (phosphate buffered saline, pH7.4)-prewashed anti-Flag G1 affinity resin (Genscript, Piscataway, NJ) at 4° C. for at least 1 hour. Then, the beads were washed with washing buffer (50 mM Tris, pH7.4, 500 mM NaCl, 0.05% Triton-X100, and protease inhibitors) 5 times of resin volume, and were eluted with 3× Flag peptide (APExBIO, Houston, TX) at a concentration of 200 μg/mL. Finally, the eluted protein was dialyzed against PBS twice and against binding buffer (B buffer: 20 mM Tris-HCl, pH 8.0. 125 mM NaCl, 10% glycerol, 1% NP-40, 5 mM DTT, and protease inhibitors) once, and was concentrated 10 times using polyethylene glycol 6000 (PEG). The concentrated protein was quantified, aliquoted, and stored at −80° C.

### Gel Shift Assay

Gel shift assay was performed essentially following a method in Christensen et al. (1995). Duplex DNA probe was generated by annealing complementary oligos, at a concentration of 45 UM, in an annealing buffer (10 mM Tris, pH 7.5, 50 mM NaCl, 1 mM EDTA) after boiling for 5 minutes. The dsDNA probe was desalted using a G-50 column (GE Healthcare). Duplex DNA probes are HBoV1 OriR (FIG. 2F), HBoV1 OriR-mut (5′-CGC GAA ACT CTA TAT CTT TTA ATG GCA GAA TTC AGC ACA TGC GCC A-3′ (SEQ ID NO:4)), B19V Ori (5′-GCC GCC GGT CGC CGC CGG TAG GCG GGA CTT CCG GTA CA-3′ (SEQ ID NO:5) (Tewary et al., 2014), and B19V Ori-mut (5′-AGC TAT TGG TCG CTA TTG GTA GGC GGG ACT-3′ (SEQ ID NO:6) (Tewary et al., 2014).

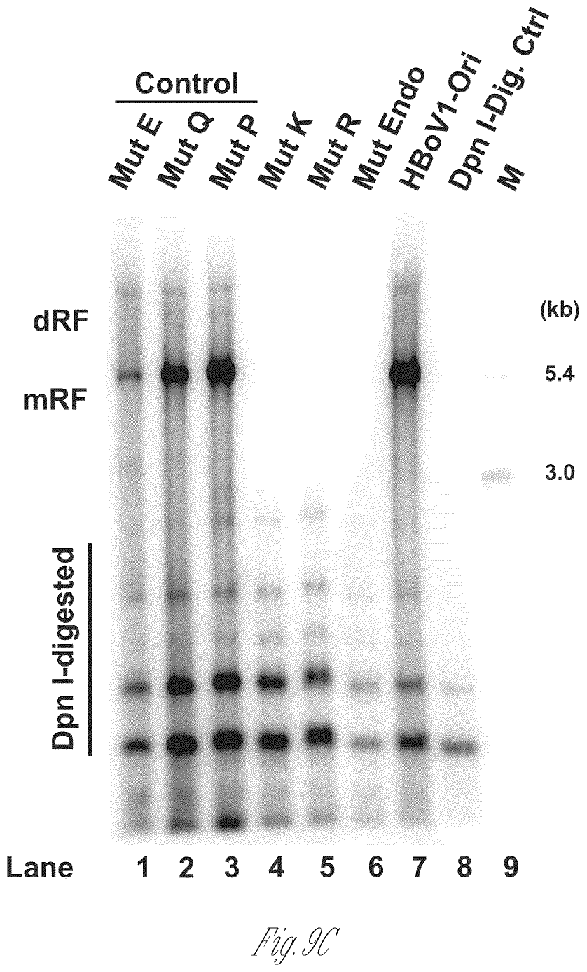

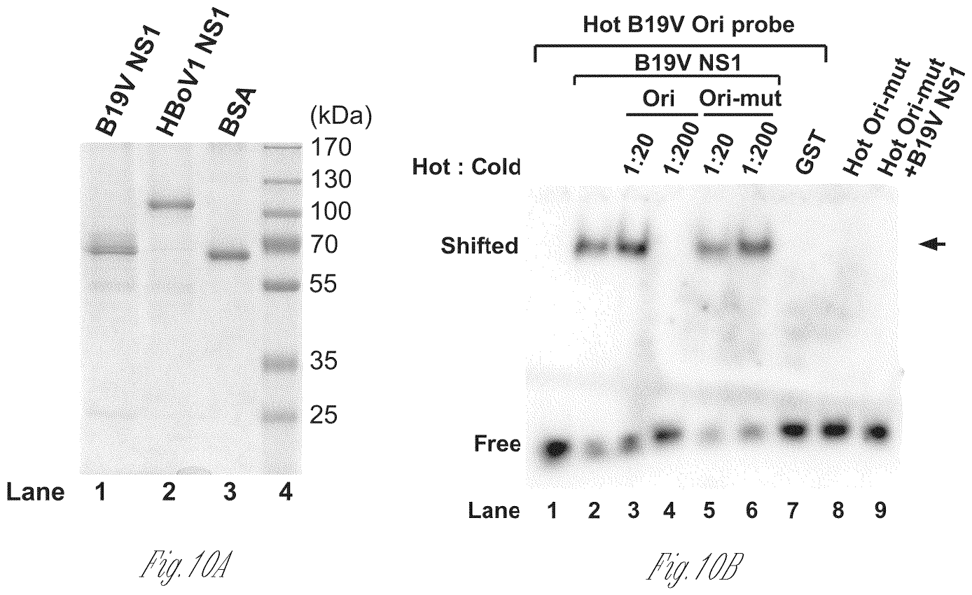

One μL of the duplex DNA probe was labeled with γ-p32-ATP using T4 polynucleotide (NEB) following the manufacturer's instructions. The binding reaction consisted of 8 μL of 2.5× B buffer, 2 μL of diluted duplex DNA probe (at 1:10,000), with/without 2 μL of purified protein (300 ng/μl), 2 L of unlabeled (cold) probe at a concentration (ratio) specified in FIG. 10, and dH₂O to a total volume of 20 μL. The reactions were incubated on ice for 20 minutes, loaded directly into a pre-run 5% non-denaturing polyacrylamide gel, and electrophoresed for 45 minutes at 100 volts. Finally, the acrylamide gel was dried by vacuum at 70° C. and exposed to a phosphor screen.

### Nuclear Extract (NE) Preparation

Nuclear extraction was performed following a method described in Dignam et al. (1983). HEK293 cells of one dish of 100 mm were transfected with pOptiHBoVINS1 or pOptiB19VNS1. At 2 days post-transfection, cells were collected, washed with ice-cold PBS, and pelleted. The cell pellet was lysed in 5× L buffer (10 mM HEPES, pH7.5, 10 mM KCl, 0.1 mM EDTA, 1 mM DTT, 0.5% NP-40, and protease inhibitors). The lysate was vortexed and centrifuged at 500 g for 5 minutes at 4° C. The pelleted nuclei were washed 3 times with 1 ml W buffer (10 mM HEPES, PH 7.5, 10 mM KCl, 0.1 mM EDTA, 1 mM DTT, and protease inhibitors). Nuclei were resuspended in 0.25 mL of NE buffer (20 mM HEPES, pH 7.5, 420 mM NaCl, 1 mM EDTA, 1 mM DTT, 25% glycerol (v/v), and protease inhibitors), and were incubated on ice for 30 minutes. Finally, nuclear extract was obtained as the supernatant by centrifuging the lysed nuclei at 12,000 g for 10 minutes at 4° C., and was adjusted the NaCl concentration to 100 mM using B1 buffer (=buffer NE without NaCl).

### Pull-Down Assay

Biotin-labeled probe was generated by annealing two complementary oligos, in which one oligo was biotinylated, at a concentration of 5 μM. Annealed dsDNA probe was desalted using a G-50 column. Streptavidin-conjugated beads (Gold Biotechnology, St. Louis, MO) were prewashed following the manufacturer's instructions. Binding reaction consisted of 200 μL of 2.5× B buffer, 100 μL of nuclear extract (about 5 μg/ml), 1 μL of biotinylated probe, with/without unlabeled probe, and dH₂O in a total volume of 0.5 ml. The reactions were rotated at 4° C. overnight, and were pelleted by centrifugation at 1,000 g for 3 minutes The pellet was then washed 3 times with cold PBS before adding loading buffer. The samples were boiled for 5 minutes and analyzed by SDS-polyacrylamide gel electrophoresis, followed by Western blotting.

### Immunoprecipitation (IP) Assay

HEK293 cells cultured in one 60 mm dish were mock-transfected or transfected with plasmids. At 2 days post-transfection, the cells were washed and lysed with 300 μL radioimmunoprecipitation assay (RIPA) buffer (50 mM Tris-HCl, pH 8.0, 150 mM NaCl, 1% NP-40, 0.5% sodium deoxycholate, 0.1% SDS, and protease inhibitors) with constant agitation for 30 minutes at 4° C. The cell lysates were centrifuged at 14,000 g for 3 minutes at 4° C., and the supernatant was collected. The rest supernatant (about 220 μL) was pre-incubated with 40 μL normal rat serum, 30 μL of prewashed protein A/G bead (ThermoFisher), and 2 μL of benzonase (ThermoFisher) for 3 hours at 4° C. with rotation After preclearing, the supernatant was equally divided for incubation with either 2 μL of normal rat IgG (0.4 mg/mL; Santa Cruz, Dallas, TX) or 20 μL of rat antisera, 30 μL of protein A/G beads, and 300 μL of RIPA buffer at 4° C. overnight. Finally, the protein A/G beads were pelleted down and washed 3 times with ice cold PBS before mixing with loading dye for Western blotting.

Results Identification of viral elements required in cis or in trans for HBoV1 replication

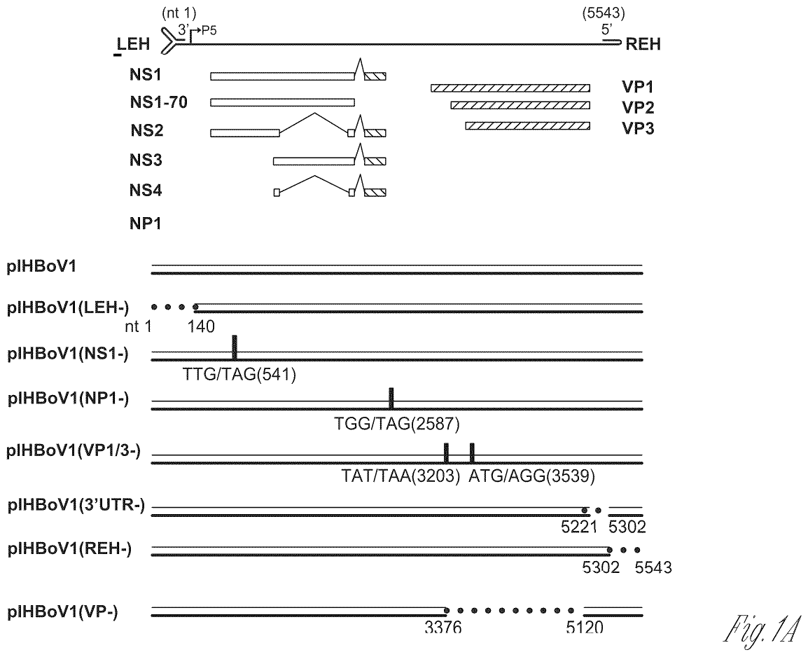

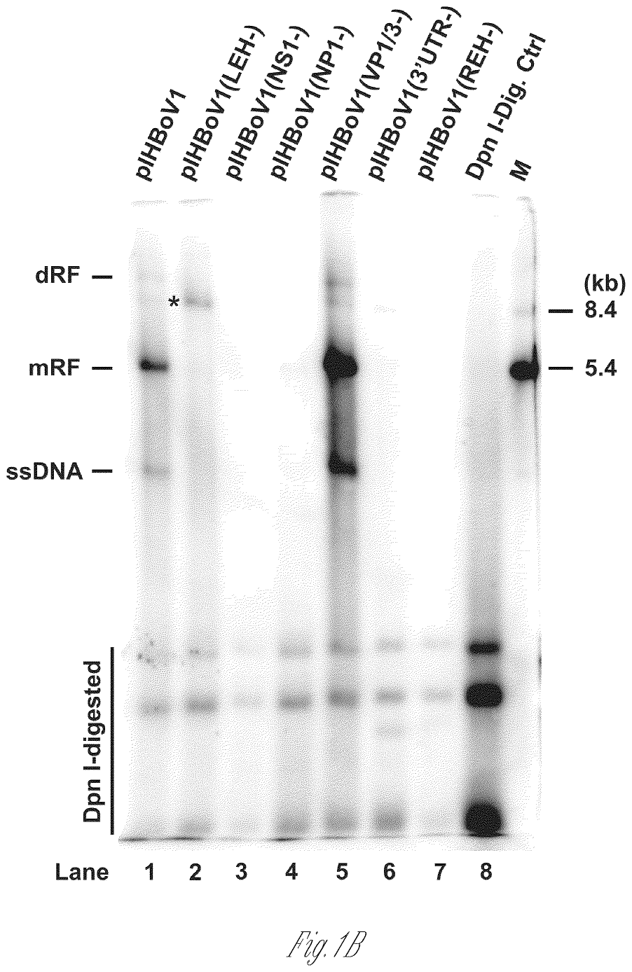

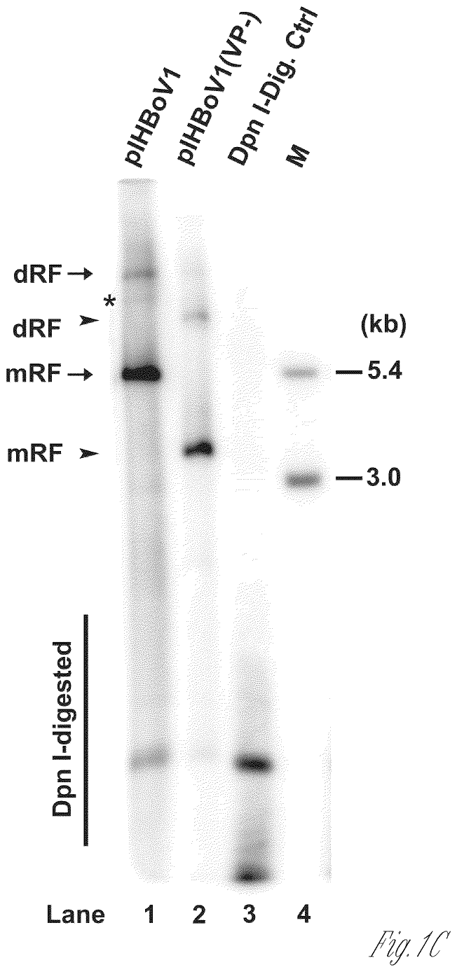

To understand the DNA replication mechanism of HBoV1, viral DNA signals and viral proteins that are required for viral DNA replication were delineated. As diagrammed in FIG. 1A, the HBoV1 genome contains four noncoding sequences (LEH, P5 promoter region, 3′ UTR, REH) and three ORFs that encode NS1-4, NP1, and VP1-3 proteins, respectively (Zou et al., 2016; Shen et al., 2015). To dissect the minimal requirements for viral DNA replication, sequential deletions of the noncoding regions and early termination of the ORFs were carried out in the context of plHBoV1. Southern blotting results showed a representative pattern of HBoV1 DNA replication including the Dpn I-digestion resistant bands: viral single-strand DNA (ssDNA, genome size), viral RF DNA and double RF (dRF) DNA, and plasmid-replicated DNA (FIG. 1B, lane 1). The results suggested that, without the LEH sequence, plHBoV1 (LEH-) still replicated but failed to excise the HBoV1 RF DNA out of the plasmid (FIG. 1B, lane 2). VP1/3-knockout plasmid plHBoV1 (VP1/3-) replicated better than the parent plHBoV1 (FIG. 1B, lanes 5 vs 1). However, plHBoV1 (NS1-), plHBoV1 (3′UTR-), and plHBoV1 (REH-) did not replicate (FIG. 1B, lanes 3, 6 and 7, respectively), and plHBoV1 (NP1-) replicated very poorly (FIG. 1B, lane 4), suggesting that the sequence at the 3′ end, including REH and 3′ UTR, and expression of NS1 and NP1 proteins were apparently important to HBoV1 DNA replication. plHBoV1 (VP-), in which the VP encoding ORFs were largely deleted, replicated as well as the plHBoV1 (FIG. 1C, lane 2).

Taken together, these results suggest that REH and 3′ UTR are largely responsible for HBoV1 DNA replication as cis-acting elements, while NS1 and NP1 serve trans-acting functions (Shen et al., 2015). In the following experiments, both LEH deletion and the VP1/3 knockout HBoV1 plasmid, plHBoV1 (LEH-VP-) were used, for further characterization of HBoV1 DNA replication at REH. Identification of the HBoV1 minimal replication origin (OriR) at REH

For DNA replication of either dependoparvovirus or autonomous parvovirus, RBEs or NSBEs and a TRS are requisite at the end palindromic hairpinned sequence (Ryan et al., 1996; Cotmore et al., 2000; Cotmore et al., 1994; Cotmore et al., 2005). These two cis-signals are necessary for Rep78/68 or NS1 to recognize the replication origin and perform strand-specific nicking, and to initiate DNA replication. To identify the minimal replication-requisite sequence on REH, a serial of truncation mutants of the REH on the base of plHBoV1 (LEH-VP 1/3-) were constructed (FIG. 2A). Linearized HBoV1 DNA was used for in vivo DNA replication analysis, in order to avoid circular plasmid DNA replication as that seen from plHBoV1 (LEH-) (FIG. 1B, lane 2). The first group of truncations contained a progressive deletion from the 3′ end of the REH (FIG. 2A, Group I). The results showed that the level of viral DNA replication progressively decreased as 3′ end nucleotides of the REH were removed (FIG. 2B). As there was a clear decrease of viral DNA replication resulting from ΔREH2 to ΔREH3 HBoV1 DNA (FIG. 2B, lanes 3 vs 4), the right end was fixed at nt 5,430 of the ΔREH2 HBoV1 DNA, and progressive truncations from the 5′ end (nt 5,344) were prepared (FIG. 2A, Group II). Results of viral DNA replication analysis showed a large decrease in RF DNA from ΔREH10 to ΔREH11 (FIG. 2C, lanes 3 vs 4). Thus, the 5′ end of the OriR was determined to be at nt 5,357, the 5′ end of ΔREH10.

To define the 3′ end of the OriR more carefully, progressive deletions from the 3′ end of ΔREH10 (nt 5,357) were determined. DNA replication analysis of the mutants from ΔREH17 and ΔREH19-24 showed that further deletions of the 3′ end after nt 5,402 (FIG. 2D, ΔREH21/lane 6) significantly decreased the level of RF DNA (FIG. 2E). This result defined the 3′ end of the OriR at nt 5.402. In addition, ΔREH18 was created, in which three nucleotides were deleted at the 5′ end in ΔREH17, to confirm the 5′ end of the OriR. The result showed that replication from ΔREH 17 was significantly decreased by about 3-fold (FIG. 2D, lane 3).

Collectively, HBoV1 OriR is defined as a 46-nt DNA of nt 5,357-5,402 (FIG. 2F). Of note.

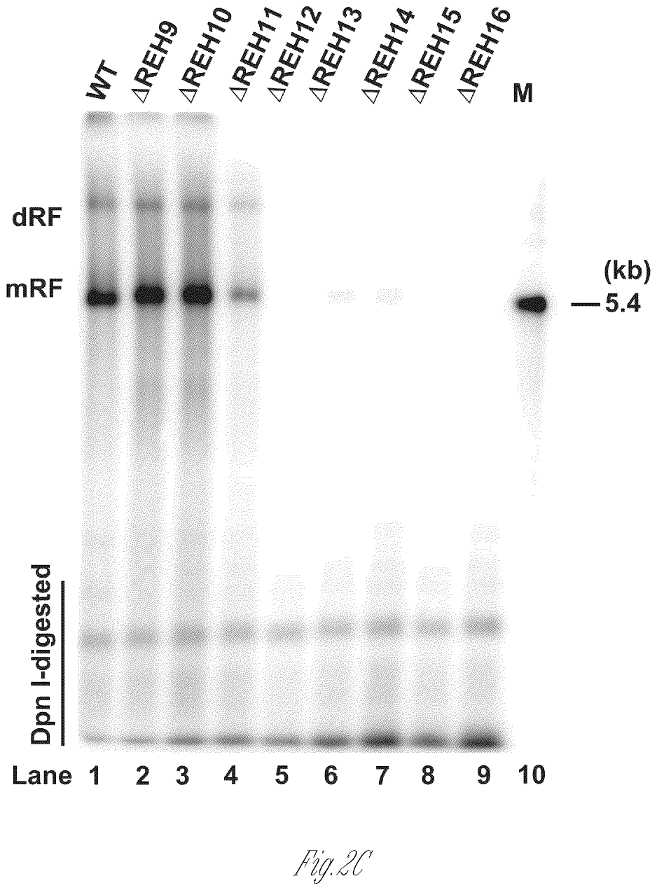

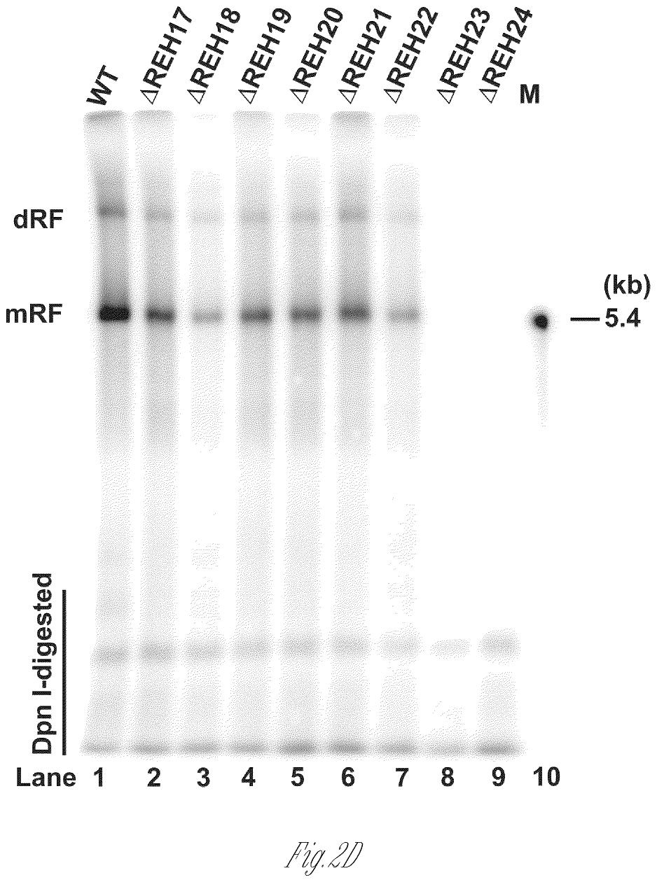

HBoV1 has a long 3′ UTR signal in front of the OriR. To clarify whether the OriR replicates viral DNA independent of the 3′ UTR, various λ DNA of 0.2, 0.4, and 1.0 kb were inserted between the 3′ UTR and the OriR in HBoV1-Ori DNA (FIG. 3A). DNA replication analysis showed that the OriR conferred viral DNA replication regardless of the insertion sizes between the 3′ UTR and OriR, supporting that the OriR functions as a template of terminal resolution independently of the adjacent 3′ UTR (FIG. 3B). NP1 colocalized with NS proteins within the APAR bodies (parvoviral DNA replication centers), and is required for terminal resolution of HBoV1 DNA

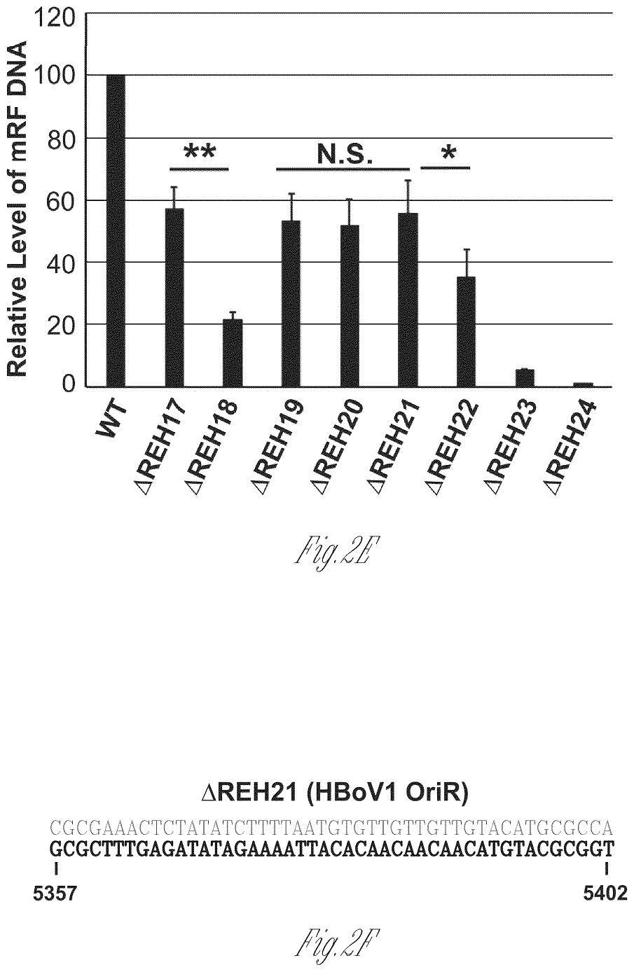

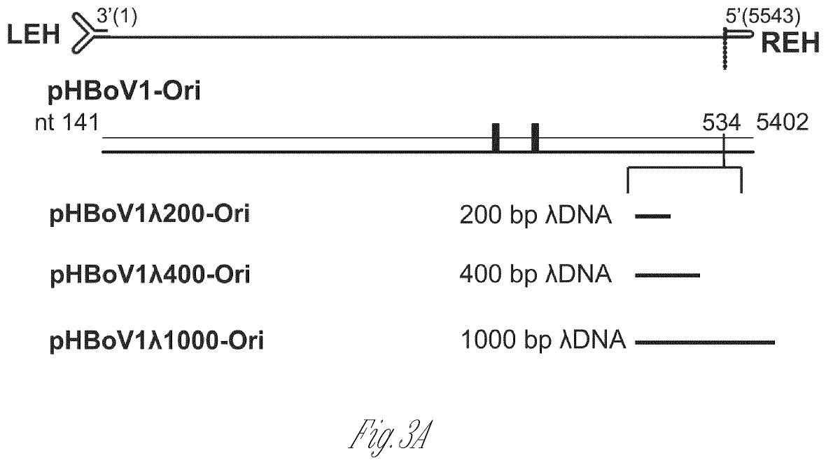

The trans-acting factors that facilitate terminal resolution at OriR were checked. NS1 or NP1 knockout plasmids based on pHBoV1-Ori were constructed, and viral DNA replication analysis performed. Without NS1/2 or NP1 expression, replication at the OriR was abolished (FIG. 4, lanes 283), reflecting that NS1 and NP1 are required for HBoV1 DNA terminal resolution, since NS2 is not required for HBoV1

### DNA Replication in HEK293 Cells (Shen et al., 2015).

Replication of parvovirus MVM and H-1 takes place in discrete subnuclear compartments, where termed autonomous parvovirus-associated replication (APAR) bodies (Bashir et al, 2001; Cziepluch et al., 2000). APAR bodies are active sites of viral DNA replication and contain cellular DNA replication factors and parvovirus NS1 (Bashir et al., 2001). The APAR bodies were located using BrdU to pulse chase active replicating viral ssDNA (Luo et al., 2013; Luo et al., 2011). Similar to other parvoviruses (Bashir et al., 2001, Cziepluch et al., 2000), the APAR bodies of HBoV1 replication showed various patterns in different cells, from foci-like dots to broad distribution in the nucleus, and colocalized with NS proteins stained with anti-NS1C in HEK 293 cells transfected with HBoV1-Ori or plHBoV1 (FIG. 5A, NS/BrdU). Notably, co-localization patterns of NP1 and BrdU staining were similarly observed for NS and BrdU co-staining, suggesting that both NP1 and NS localized within APAR bodies (FIG. 5B, NP1/BrdU). In support of this hypothesis, NS and NP1 proteins colocalized in the nucleus of HBoV1-Ori or plHBoV1-transfected cells (FIG. 5C, NP1/NS) More importantly, in HBoV1-infected HAE cells, NS and NP1 co-localized well within the BrdU-chased APAR bodies (FIG. 5D-F). To confirm the localization of NP1, a proximity ligation assay (PLA) was used to visualize interactions of NP1 with BrdU-labeled viral ssDNA or dsDNA/ssDNA intermediates. Clearly positive fluorescent foci were observed in the nucleus of pHBoV1-Ori, plHBoV1-transfected, or HBoV1-infected cells stained with anti-NP1 and anti-BrdU antibodies (FIG. 5G). PLA shows bright signals if the two molecules localize proximately at a distance of about 20 nm (Soderberg et al., 2006).

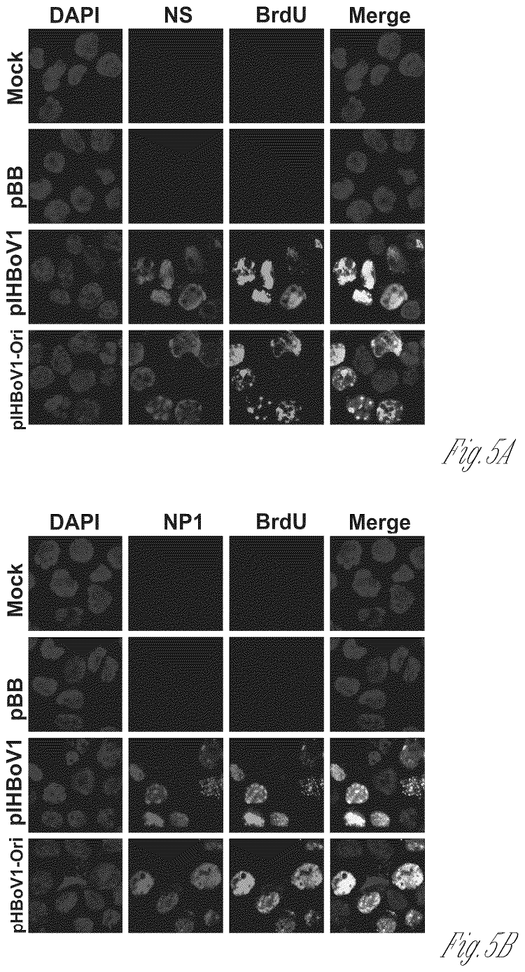

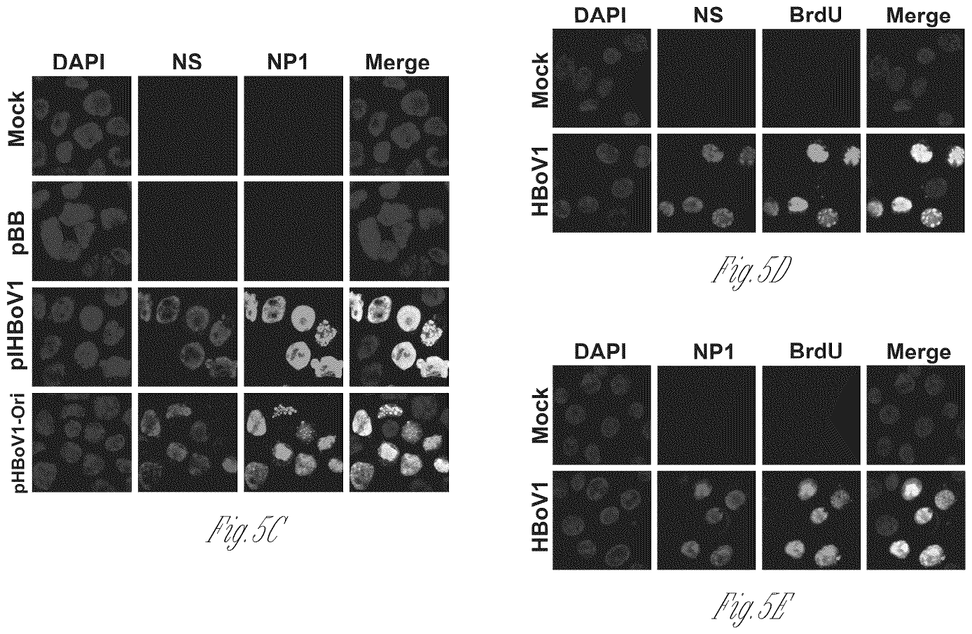

To explore a direct interaction between NS1 and NP1, immunoprecipitation assays were performed. Cells transfected with pHBoV1-Ori were lysed and pre-cleared with normal rat sera. Pre-clear lysates were immunoprecipitated with control rat IgG or anti-NP1 serum in the presence of nuclease treatment, and were then evaluated for NS1 by Western blotting. Neither NS1, NS2, NS3 nor NS4 were co-immunoprecipitated with NP1 (FIG. 6A, lanes 6 vs 4). As a control, using an anti-NS1C antibody, all NS proteins could be immunoprecipitated (FIG. 6B, lanes 3 vs 1). These results suggest that NS1-4 and

### NP1 Proteins do not Directly Interact.

Taken together, these results suggest that either by transfection of plHBoV1/plHBoV1-Ori or during viral infection, HBoV1 NS1-4 and NP1 proteins function synergistically in the viral DNA replication centers (APAR bodies) but without a direct interaction. More importantly, these results confirm a direct involvement of the HBoV1 NP1 during viral terminal resolution at OriR.

Identification of the NS1 Binding Elements (NSBEs) and TRS within OriR

The replication origins of parvoviruses harbor multiple binding elements of Rep78/68 or NS1 and a site-specific TRS (Ryan et al., 1996; Cotmore et al, 2000; Cotmore et al., 1994; Cotmore et al., 2005). Therefore, NSBE and TRS were identified by means of mutagenesis. The NSBEs characterized in AAV and MVM by both in vivo and in vitro studies are several tetra-nucleotides repeats, which are directly recognized by the origin binding domain (OBD) of the AAV Rep68/78 or MVM NS1. Notably, there are no cognate NSBE for HBoV1 NS1 binding that can be located in both REH and LEH (Huang et al., 2012). In contrast, similar to the 4 repeats of tri-nucleotides in Galleria mellonella densovirus (GmDNV), there are 4 repeats of “TGT” tri-nucleotides “5′-TGT TGT TGT TGT-3′ (SEQ ID NO:7)” in the OriR (FIG. 7A) (Ding et al., 2002).

Serial mutations were made in the “TGT” repeated region in the OriR, and performed in vivo DNA replication analysis. The results showed that all these mutations of the “TGT” tri-nucleotides significantly decreased DNA replication to various levels under 40% of the activity of the wild-type (FIG. 7B&C), however, the mutation of multiple “TGT” progressively disable DNA replication to a greater extent. For example, mutating two “TGT” retained DNA replication better than mutating three “TGT” (FIG. 7B&C, lanes 1&2 vs lanes 4-7). Mutating all four “TGT” decreased DNA replication the most, at level of only 5% of the wild-type (FIGS. 7B and C, lane 3). Thus, these results suggest that this “TGT” repeating sequence is likely the HBoV1 NSBEs.

The nicking sites of parvoviruses are not conserved (Cotmore et al., 2015). In principle, after nicking, the Ori reveals transient nicked intermediate (ssDNA breaks) before the free 3′-hydroxyl (OH) extends by DNA polymerase (Cotmore et al., 2005). A strategy of rapid amplification of the nicked 3′ end was used to characterize the intermediates of the resolved OriR after NS1 nicking. Using Hirt DNA extracted from plHBoV1- or HBoV1-Ori-transfected cells, adenosines were added to extend the 3′ end with polyA residues. With subsequent PCR amplification and cloning, we mapped a few transient ending sites in the OriR (FIG. 8A). To identify the nicking among these ending sites, each site located outside of the NSBEs was mutated (FIG. 8A). Analysis of in vivo DNA replication showed that mutations at either the T or A nucleotide at nt 5,368 or 5,369, but not the mutations in other locations, completely abolished viral DNA replication, (FIG. 8B, lanes 586 vs lanes 1-4). A mutation at nt 5,373 within the putative nicking motif (5′-CTA TAT CG-3′ (SEQ ID NO:8) also reduced the level of RF DNA (FIG. 8A, TRSm7, and FIG. 8B, lane 7). This result defined the T at nt 5,368 as the nicking site (5′-CTA/TATCT-3′ (SEQ ID NO: 9)).

### Identification of the Key OriR-Binding Residues in the Origin-Binding Domain (OBD) of the HBoV1 NS1

The structure of the HBoV1 OBD has been resolved, and it was predicted that NS1 utilizes two non-structured positively charged loop regions to bind Ori (Tewary et al., 2013). The binding residues of the NS1 were examined using a mutagenesis approach (FIG. 9A). To this end, mutations of either amino acids at the positively charged loops (loop K: ¹²⁷KR¹²⁸ and loop R: ¹⁹³RR¹⁹⁴) or predicted endonuclease activity motif of the NS1 (¹¹⁵HCH¹¹⁷), together with three control mutations (Mut Q. P. and E) were performed, in the context of HBoV1-Ori. Controls Mut Q and Mut P mutated putative loop K and loop L, respectively. Mut E control mutated the nearby amino acids (¹²³EGL¹²⁵) of putative loop K (FIG. 9B). Structures of the mutated NS1 OBD were predicted based on the wild-type NS1 OBD (Tewary et al., 2013). Superimposition forms of mutants with wild-type NS1 OBD showed that the mutagenesis did not significantly change the structure (FIG. 9B). In vivo DNA replication analysis showed that mutating either the basic charged loop region (Mut K and Mut R) or the endonuclease core (Mut Endo) abolished replication, whereas the three control mutations did not alter DNA replication very much (FIG. 9C). These data suggest that the two positively charged loops of the OBD, as well as the two histine residues at the predicted endonuclease motif, are required for HBoV1 DNA replication, and that the two positively charged loops likely play a significant role in the NS1 and OriR binding.

### HBoV1 NS1 Did not Specifically Bind OriR In Vitro

To understand the interaction between HBoV1 NS1 and OriR (FIG. 2F). HBoV1 NS1 (FIG. 10A, lane 2) was purified and studied its binding with the OriR studied in vitro. As a positive control for NS1 binding to OriR. B19V NS1 was purified (FIG. 10A, lane 1) and carried along in parallel. Electrophoresis gel shift assay showed that B19V NS1 shifted the B19V Ori (FIG. 10B, lane 2). This binding is specific because it was competed by excess cold B19V Ori but not by excessed cold mutated B19V Ori (Ori-mut) (FIG. 10B, lanes 4 vs 6). As a negative control, GST protein did not shift the B19V Ori (FIG. 10B, lane 7), and the B19V On-mut was not shifted by B19V NS1 (FIG. 10B, lanes 8 vs 9). In contrast, however, with the same experimental condition of the electrophoresis gel shift assay, HBoV1 NS1 did not bind its own OriR specifically (FIG. 10C, lane 2). Adding either cold OriR (FIG. 10C, lanes 3-5) or cold Ori-mut (FIG. 10C, lanes 6-8) showed similar negative signals. Of note, weak non-specific interaction were found between HBoV1 NS1 and non-specific DNA in the binding buffer without poly [d (I: C)] (FIG. 10D, lane 2). This non-specific binding was competed either by excessed cold HBoV1 OriR (FIG. 10D, lanes 3-5) or cold HBoV1 OriR-mut (FIG. 10D, lanes 6-8). Thus, the gel shift assays suggest that HBoV1 NS1 alone does not bind HBoV1 OriR specifically in vitro.

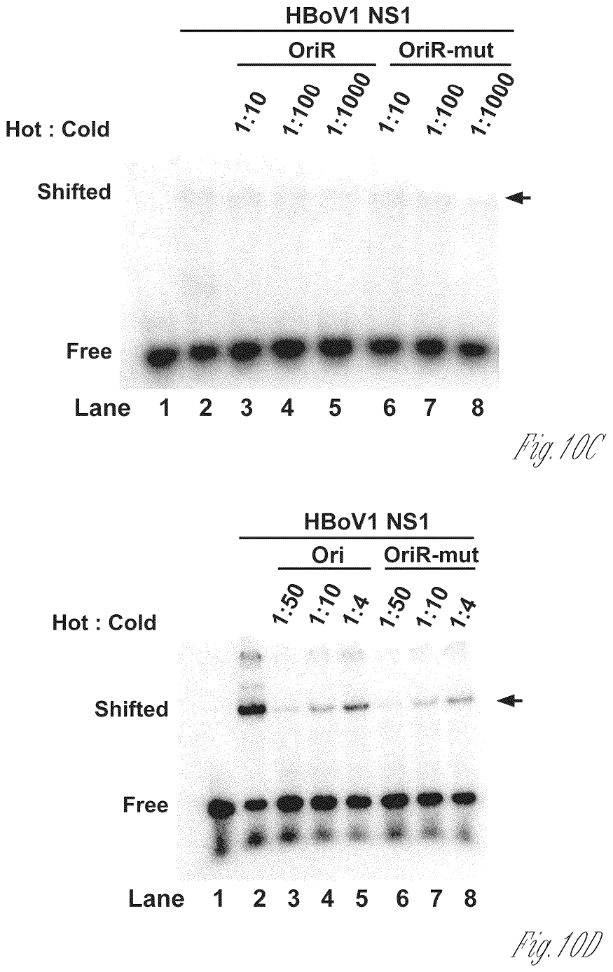

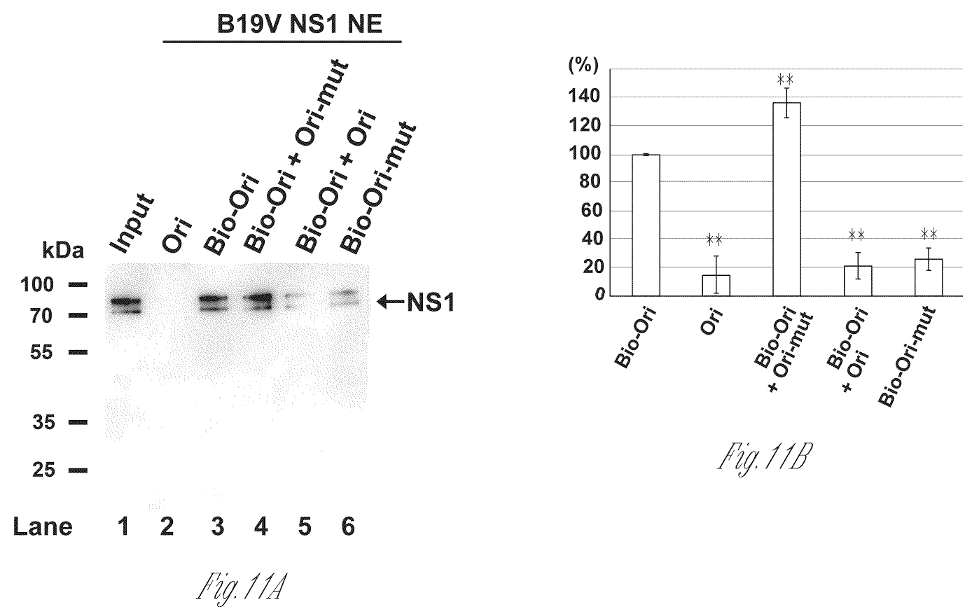

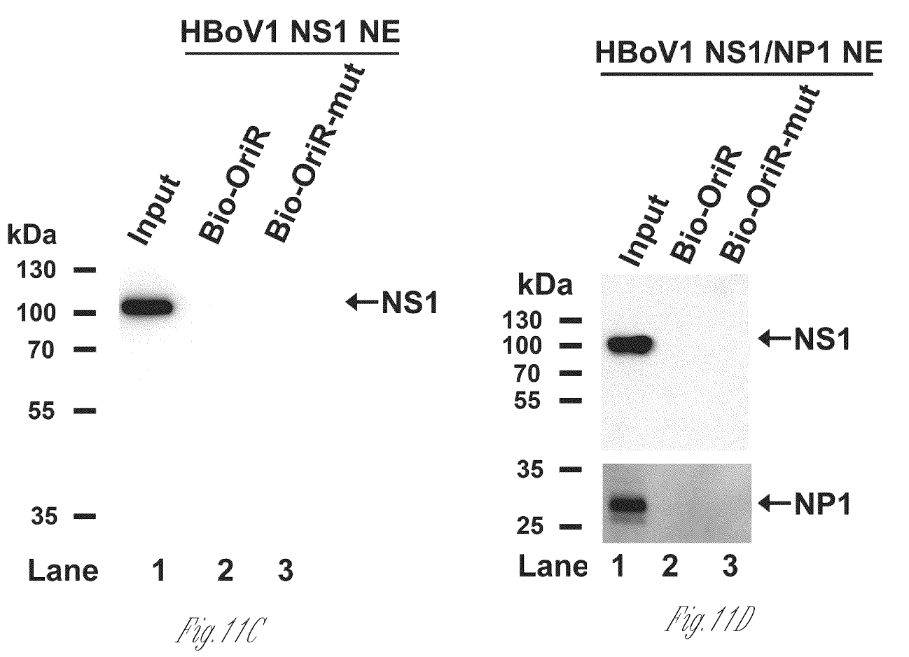

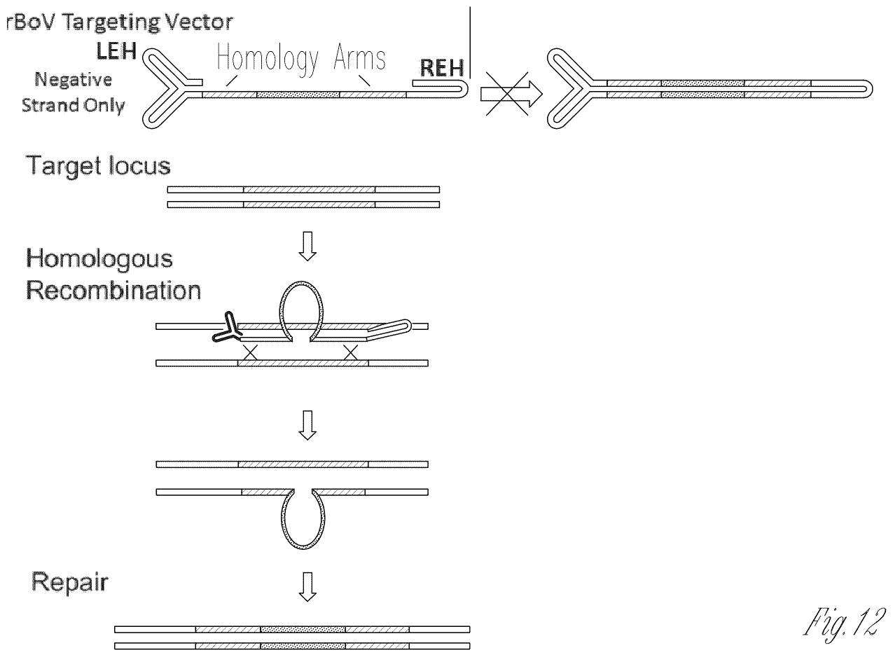

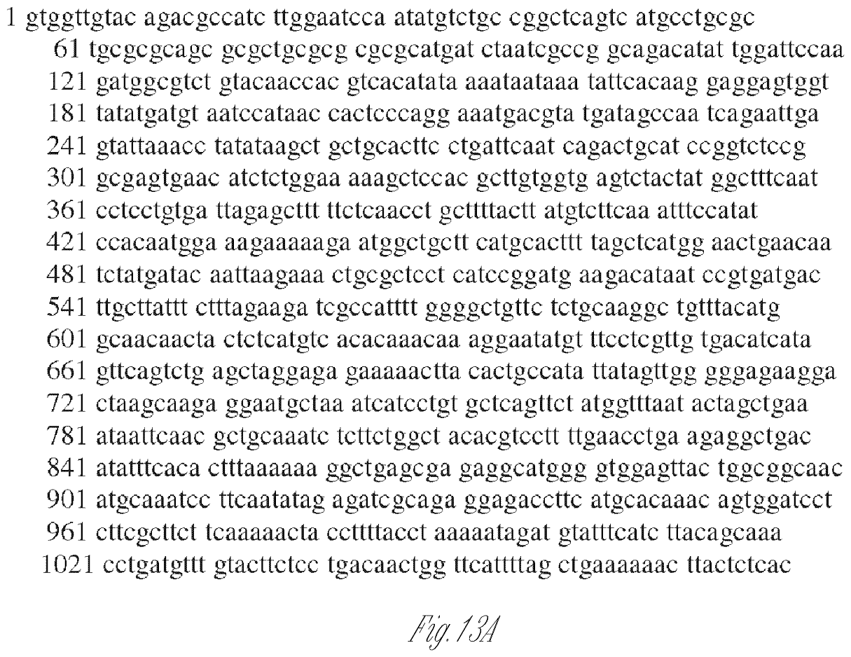

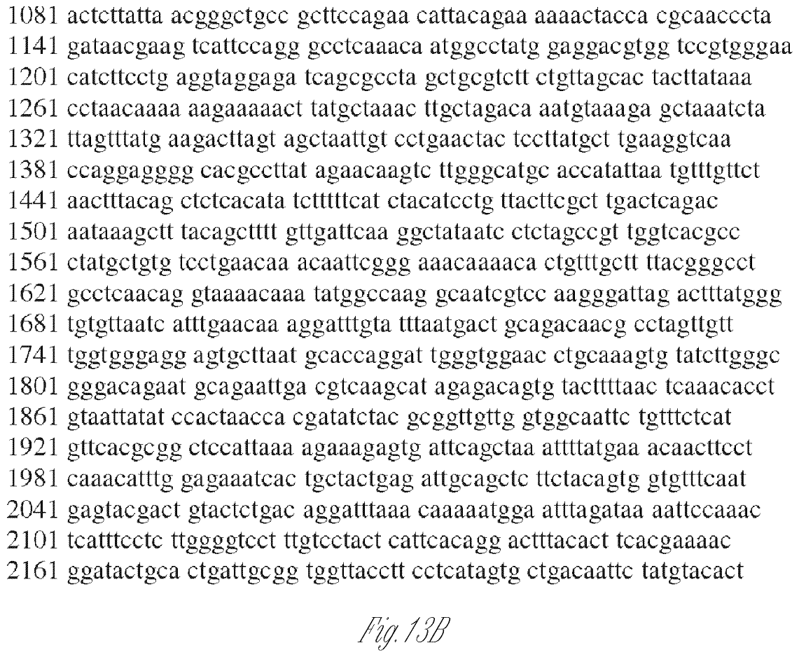

To further examine the binding property between HBoV1 NS1 and OriR in the presence of cellular proteins, a biotin pull-down assay was designed. Nuclear extract prepared from NS1-transfected HEK 293 cells was used for pull-down by biotin-labeled OriR (Bio-OrR) and streptavidin-conjugated beads, and then analyzed by Western blotting. In the experiment with B19V Ori, biotinylated B19V Ori (Bio-Ori) pulled down B19V NS1, which was competed by excess B19V Bio-Ori but not by excess biotinylated mutant Ori (Bio-Ori-mut) (FIG. 11A, lanes 4 vs 5, and FIG. 11B). As a control, non-biotin-labeled B19V Ori did not pull down any B19V NS1 (FIG. 11A, lane 2, and FIG. 11B), and Bio-Ori-mut only pulled down a few minor amount of NS1 (FIG. 11A, lane 6, and FIG. 11B). By contrast, HBoV1 NS1 was not pulled down by either biotinylated Ori or Ori-mut (FIG. 11C, lanes 2&3), despite high-level expression of NS1 (FIG. 11C, lane 1). As NP1 co-localized with BrdU labeled viral replication centers (FIG. 5), the hypothesis was tested that NP1 is important for NS1 binding with OriR. Nuclear extracts were prepared from cells co-expressing NP1 and NS1, and performed pull-down assays. The results demonstrated that biotinylated HBoV1 OriR did not pull down NS1, even in the presence of NP1 (FIG. 11D, lane 2). Collectively, the pull-down assays suggest that HBoV1 NS1 does not bind HBoV1 OriR in vitro in the presence of cellular factors and NP1.

### DISCUSSION

In this report, the viral components that are important for cis and trans HBoV1 terminal resolution at REH were studied. A 46-nt sequence at nt 5,357-5,402 was defined as the HBoV1 OriR. It contains both the NSBEs and TRS, and is used as a template for terminal resolution of HBoV1 DNA. Notably, a cis sequence in the 3′ UTR was important for terminal resolution, while the large NS protein NS1 and the small NP1 played a pivotal role in trans for terminal resolution. These basic findings of HBoV1 terminal resolution at the OriR lay a foundation for further understanding the mechanism underlying NS1 binding to and nicking at the OriR, which are the key steps in HBoV1 RF DNA replication. Additionally, in this study, the specific binding of the B19V NS1 with B19V Ori was shown in vitro.

### Functions of Viral Proteins in HBoV1 Terminal Resolution at OriR

NS1 function: The large non-structural protein of parvovirus, Rep78/68 or NS1, is composed of a putative DNA origin binding/endonuclease domain (OBD), helicase activity domain, and transactivation domain (TAD) at N-terminus, middle, C-terminus, respectively (Shen et al., 2015). The crystal structure of the HBoV1 OBD was resolved at 2.7-A resolution, which is similar to the canonical histidine-hydrophobic-histidine superfamily of nucleases. The OBD structure combines two distinct functions: (i) a positively charged region formed by surface β-hairpin (aa190-198) and helix a5 (aa127-132), which is responsible for recognizing the viral DNA replication origin, and (ii) the endonuclease active site which contains signature motif HUH and performs strand-specific cleavage at Ori (Tewary et al., 2013). The HUH motif of HBoV1 OBD contains two histidine residues (H115/H117) separated by cysteine C116, followed by the three hydrophobic residues 1118, L119, and V120 (Tewary et al., 2013). However, these active sites are only predicted from superposition of the HBoV1 OBD structure into the AAV5 OBD structure (Hickman et al., 2004) Here, it was confirmed that R193 and R194 in the surface hairpin of the OBD and K127 and R128 in the loop region are critical to terminal resolution, and, by contrast, the neighboring 123EGL¹²⁵ residues are not. Mutation of the two histidine residues (H115 and H117) confirmed their nicking function. Structure study of the MVM NS1 OBD also revealed conserved residues of DNA binding and nicking activities (Tewary et al., 2015), highlighting the importance of the confirmed DNA binding and nicking residues of the HBoV1 NS1 OBD.

NP1 function: Previously it was shown that bocaparvovirus NP1 plays an important role in the replication of viral duplex DNA (Sun et al., 2009; Huang et al., 2012). HBoV1 NP1 or BPV1 NP1 can complement replication of a mutant MVC infectious clone that does not express MVC NP1 (Sun et al., 2009). Three other newly identified HBoV1 small NS proteins NS2-4 are not required for replication of HBoV1 duplex DNA genome in HEK293 cells (Sun et al., 2009) Since NS2-4 all contain the NS1C terminus, the anti-NS1C antibody reacts with all these isoforms (Sun et al., 2009). Thus, it was demonstrated that all NS1-4 proteins and NP1 colocalize within APAR bodies. Nevertheless, it is unlikely that NS2-4 recruit NP1 to the APAR bodies. Direct interactions between NS1-4 and NP1 were not confirmed (FIG. 6). NP1 contains a non-classical nuclear localization signal (ncNLS) at aa7-50 (Li et al., 2013), and is able to complement the functions of the MVM NS2 in viral DNA replication during an early phase of infection (Mihaylov et al., 2014). In fact, during MVM infection of NP1-expressing A9 cells, NP1 was progressively lost from its nucleolus-localization and began to be colocalized with MVM NS1 in APAR bodies. Moreover, NP1 expression rescues APAR body maturation in cells infected with an NS2null mutant of MVMp (Mihaylov et al., 2014). Additionally, NP1 is involved in viral pre-mRNA processing. HBoV1 NP1 is required for viral mRNA splicing at the A3 splice site and read-through of the viral mRNA through the (pA) p site. Taken together, it was hypothesize that during infection or viral DNA replication, NP1 is critical in the development of viral DNA replication centers (APAR bodies) together with NS1, in which cellular DNA replication factors are enriched; however, after the formation of the APAR bodies, NP1 could be relocated to the cellular compartment for viral RNA processing. On the other hand, NP1 could possibly be recruited by cellular DNA replication factors to the APAR bodies, in response to the efforts of NS1 in interacting with the viral Ori and cellular DNA replication factors (Cotmore et al., 1995; Nash et al., 2009; Ihalainen et al., 2007).

### Identification of the HBoV1 OriR

For a homotelomeric parvovirus whose replication depends on a helper virus, a 43-nt DNA sequence, was identified as the AAV Ori, containing the Rep binding element (RBE) and TRS (Ryan et al., 1996) For homotelomeric parvoviruses that replicate autonomously, a specific 38-nt DNA sequence has been identified as the Ori of goose parvovirus (GPV) (Smith et al, 1996), and a 67-nt DNA was identified as B19V Ori that contains a TRS and four GC-rich NSBEs that are required for optimal virus replication (Guan et al., 2009). The B19V ITR resembles that of GPV in that both have an arrow-like hairpin structure (Cotmore et al., 2005; Zadori et al., 1995). For heterotelomeric parvoviruses that replicate autonomously, there are two replication origins located at the LEH and REH, respectively. The active form of MVM LEH Ori (OriLre) is about 50-nt in length, composed of a transcription factor PIF binding site, the (ACCA) 2 NSBE, and the TRS site (Cotmore et al., 2005; Cotmore et al., 1994). It functions as a template for junction resolution that generates ssDNA genome. By contrast, the MVM REH Ori (OriR) is around 125-nt in length, and contains a region composed a TRS and two closely contacted (ACCA) 2 NSBEs, a degenerate NSBE (CGGT) at the tip of the hairpin, and a cis sequence that is non-specifically bound by HMG1/2 family DNA binding proteins (Cotmore et al., 2000). Therefore, the MVM OriR includes almost the entire sequence of the REH (Cotmore et al., 2000; Cotmore et al., 2005) In this study, a 46-nt sequence (OriR) at the REH of the HBoV1 genome was identified that is responsible for the replication of duplex HBoV1 genome in HEK293 cells. This OriR represents the first Ori in members of heterotelomeric parvoviruses that functions as a template of terminal resolution in a short closely contacted DNA sequence (46-nt), containing the NSBEs and TRS. That cis-acting sequences surrounding the TRS and NSBEs may be required for interacting with cellular factors, e.g., HMG1/2 with MVM OriR (Cotmore et al., 2000) and PIF with MVM OriL (Christensen et al., 1997).

### Characterization of the TRS and NSBEs

The TRS at which Rep78/68 or NS1 nicks is specific to each parvovirus (Cotmore et al., 2015). The TRS (5′-GAGT/TGG-3′) is conserved only in AAV1-4 and 6, but AAV5 uses 5′-AGTG/TGGC-3′ (Chiorini et al., 1999) For autonomous parvoviruses, GPV uses (5′TGAG/TCT3′) (Smith et al., 1999); B19V Ori uses a unique nicking site (5′-GACA/CCA-3′) (Guan et al., 2009); and MVM uses a nicking site (5′-CTWW/TCA-3′, W=A/T) (Smith et al., 1999). Thus, the nicking sites of autonomous parvoviruses differ from each other. The HBoV1 TRS (5′-CTA/TATCT-3′) identified in this study closely resembles the MVM TRS with an A/T-rich sequence in the center. Of note, such a similar TRS signal is not found at the LEH of the HBoV1 genome. However, one unique TRS of AAV2 Rep78/68 (5′-CTCCA/TTT-3′) has been identified in the minimal replication origin present within the AAV2 P5 promoter (Francois et al., 2005; Wang et al., 1997). The nicking of the AAV2 Rep78/68 at the TRS in the P5 promoter involves the TATA box in cis and the TATA-binding protein in trans (Francois et al., 2005). HBoV1 NS1 may employ a different TRS to perform junction resolution at the OriL of the LEH, and that cellular transcriptional factors or DNA binding proteins should facilitate nicking of the NS1 at the TRS at the OriL.

Several Rep78/68 and NS1 binding elements have been characterized, and confirmed by in vitro binding assay. The AAV RBE consists of three tetramer repeats (GCTC)₄ plus a degenerate GCGC (Ryan et al., 1996). Similar three tetramer repeats (GTTC) 3 plus GAAC were found in the ITR of GPV (Smith et al., 1999). Two hexamer repeats (GCCGCCGG) ¿ were confirmed to bind B19V NS1 OBD in an in vitro binding assay (Tewary et al., 2014). MVM NSBE in either the OriL or OriR, comprise 2-3 tandem copies of the tetranucleotide (TGGT)₂₋₃. Densoparvovirus GmDNV NS1 binds a (GAC) 4 trimer repeated sequence in its ITR (Ding et al., 2002). A consensus NSBE for HBoV1 NS1 binding cannot be found in the LEH and REH of HBoV1 genome. In the HBoV1 OriR identified in this study, at about 12 nt downstream of the TRS, a tetramer tri-nucleotide [(TGT)₄] was proved important to viral duplex DNA replication at the OriR. However, a specific binding between HBoV1 NS1 and OriR could not be confirmed in vitro. Although in vitro binding assay is capable of revealing the specific binding. An in vitro binding assay which has been successfully used to confirm MVM NS1 binding to its NSBE (Christensen et al., 1995) is capable of revealing the specific binding and was adapted. B19V NS1 binding to the B19V Ori was demonstrated in side-by-side studies assessing HBoV1 NS1 binding of the HBoV1 OriR. Although a strong and specific binding between B19V NS1 and its Ori was observed and confirmed, no specific binding between HBoV1 NS1 and OriR was observed. Therefore, HBoV1 NS1 and OriR binding may require the involvement of cellular proteins. However, in a subsequent in vitro pull-down assay, the biotinylated HBoV1 OriR did not pull down any HBoV1 NS1 from the lysate of the cells expressing HBoV1 NS1. As a control, B19V Ori pulled down NS1 at a significantly higher level than the mutant Ori, which can be competed by the wild--type B19V Ori but not the mutant Ori. A further experiment using nuclear extract prepared from NS1- and NP1-coexpressing cells also did not show any binding between HBoV1 NS1 and OriR

Nevertheless, based on the model of rolling circle replication in which Rep78/68 or NS1 has to bind the origin and meit the duplex viral DNA, and perform nicking of the ssDNA at the TRS of about 20-nt upstream of the NSBEs (Cotmore et al., 2005), the (TGT)₄ repeat in HBoV1 OriR may be the NSBEs. The (TGT) A repeat closely resembles the (GAC)₄ repeat of GmDNV NSBE (Ding et al., 2002). Since the (TGT)₄ repeat contains the (TGTT)₂ repeat, the HBoV1 NSBEs also resembles MVM NSBEs (TGGT)₂₋₃ (Cotmore et al., 995). Considering no cognate binding sequence can be found in the origins at both the LEH and REH of HBoV1 genome, contrary to what has been observed, HBoV1 NS1 may bind the origins at a low affinity and require the help of other viral components and cellular proteins to do so.

### Function of the 3′ UTR of HBoV1

Identification of a role of the 3′ UTR between VP-coding region and the REH in HBoV1 DNA replication is unexpected. In other parvoviruses, various cis-sequences that are outside of the terminal hairpins have been identified to be important for DNA replication, e.g., an additional AAV2 minimal DNA replication origin at the P5 promoter (5′ UTR) (Wang and Srivastava, 1997; Tullis and Shenk, 2000; Nony et al., 2001). In MVM, it has been shown that specific elements inboard of the REH between nt 4,489-4,636 and nt 4,636-4,695 are necessary for efficient replication of MVM duplex DNA (Tam and Astell, 1993). In the development of recombinant MVMp vector (rMVMp), the rMVMp genome, a large portion of cis-element was remained at the 3′ end (nt 4,631-5,149) (Hendrie et al., 2003). However, how these cis-elements outside of the hairpins facilitate viral DNA replication has not been studied.

Yan et al. (1993) developed a recombinant HBoV1 vector, in which both the rAAV2 genome and HBoV1 genome were used, however, a large portions were retained at the 3′ and 5′ end in the rHBoV1 genome, in order to ensure efficient replication in the presence of a packaging plasmid. Unfortunately, the cis-sequences that remained resulted in a high rate of recombination that generated wild-type virus in the rHBoV1 preparations (Yan et al., 2013). Therefore, to define the cis minimal requirement for HBoV1 DNA replication is important to develop a better rHBoV1 vector that may hold benefits for gene targeting in human airways, since 95% of the HBoV1 genome is negative sense, while the AAV genome has equal polarity (Sun et al., 2009, Berns, 1990). We plan to further define the cis element at the left end in order to construct an rHBoV1 genome that has a minimal sequence of HBoV1 (to avoid homology recombination with the HBoV1 packaging plasmid).

### REFERENCES

Allander, T., M T. Tammi, M. Eriksson, A. Bjerkner, A. Tiveljung-Lindell, and B. Andersson. 2005 Cloning of a human parvovirus by molecular screening of respiratory tract samples. Proc. Natl. Acad. Sci. U.S.A 102:12891-12896.
Allander, T., T. Jartti, S. Gupta, H. G. Niesters, P. Lehtinen, R. Osterback, T. Vuorinen, M. Waris, A. Bjerkner, A. Tiveljung-Lindell, B. G. van den Hoogen, T. Hyypia, and O. Ruuskanen. 2007. Human bocavirus and acute wheezing in children. Clin. Infect Dis. 44:904-910.
Bashir, T., J. Rommelaere, and C. Cziepluch. 2001. In vivo accumulation of cyclin A and cellular replication factors in autonomous parvovirus minute virus of mice-associated replication bodies. J. Virol. 75:4394-4398.
Berns, K. I. 1990. Parvovirus replication. Microbiol. Rev. 54:316-329
Brodzinski, H. and R. M. Ruddy 2009. Review of new and newly discovered respiratory tract viruses in children. Pediatr Emerg.Care. 25:352-360.
Chen, A. Y., F. Cheng, S. Lou, Y. Luo, Z Liu, E. Delwart, D. Pintel, and J. Qiu. 2010. Characterization of the gene expression profile of human bocavirus. Virology. 403:145-154.
Cheng, W. X., J. S. Li, C. P. Huang, D. P. Yao, N. Liu, S. X. Cui, Y. Jin, and Z. J. Duan. 2010. Identification and nearly full-length genome characterization of novel porcine bocaviruses. PLoS.ONE. 5: e13583.
Chiorini, J. A., S. Afione, and R. M. Kotin. 1999. Adeno-associated virus (AAV) type 5 Rep protein cleaves a unique terminal resolution site compared with other AAV serotypes. J Virol. 73:4293-4298.
Christensen, J., S. F. Cotmore, and P. Tattersall. 1995. Minute virus of mice transcriptional activator protein NS1 binds directly to the transactivation region of the viral P38 promoter in a strictly ATP-dependent manner. J.Virol. 69:5422-5430.
Christensen, J., S. F. Cotmore, and P. Tattersall. 1997 Parvovirus initiation factor PIF: a novel human DNA-binding factor which coordinately recognizes two ACGT motifs. J. Virol. 71:5733-5741.
Cotmore, S. F. and P. Tattersall. 1994. An asymmetric nucleotide in the parvoviral 3′ hairpin directs segregation of a single active origin of DNA replication. EMBO J. 13.4145-4152.
Cotmore, S. F. and P. Tattersall. 2005. A rolling-haipin strategy: basic mechanisms of DNA replication in the parvoviruses, p. 171-181. In: J. Kerr, S. F. Cotmore, M. E. Bloom, R. M. Linden, and C. R. Parrish (eds.), Parvoviruses. Hoddler Arond, London.
Cotmore, S. F. and P. Tattersall. 2005. Structure and Organization of the Viral Genome, p. 73-94. In: J. Kerr, S. F. Cotmore, M. E. Bloom, R. M. Linden, and C. R. Parrish (eds.), Parvoviruses. Hodder Arnold, London.
Cotmore, S. F., J. Christensen, and P Tattersall. 2000. Two widely spaced initiator binding sites create an HMG1-dependent parvovirus rolling-hairpin replication origin. J. Virol. 74:1332-1341.
Cotmore, S. F., J. Christensen, J. P. Nuesch, and P. Tattersall. 1995. The NS1 polypeptide of the murine parvovirus minute virus of mice binds to DNA sequences containing the motif [ACCA]2-3. J. Virol. 69.1652-1660.
Cotmore, S. F., M. Agbandje-Mckenna, J. A. Chiorini, D. V. Mukha, D. J. Pintel, J. Qiu, M. Soderlund-Venermo, P. Tattersall, P. Tijssen, D. Gatherer, and A. J. Davison. 2014. The family Parvoviridae. Arch. Virol. 159:1239-1247.
Cziepluch, C., S. Lampel, A. Grewenig, C. Grund, P. Lichter, and J. Rommelaere. 2000. H-1 parvovirus-associated replication bodies: a distinct virus-induced nuclear structure. J. Virol. 74:4807-4815.
Deng, X., Z Yan, F Cheng, J. F. Engelhardt, and J. Qiu. 2016. Replication of an Autonomous Human Parvovirus in Non-dividing Human Airway Epithelium Is Facilitated through the DNA Damage and Repair Pathways. PLoS.Pathog. 12: e1005399.
Dignam, J. D., R. M. Lebovitz, and R. G. Roeder. 1983. Accurate transcription initiation by RNA polymerase II in a soluble extract from isolated mammalian nuclei. Nucleic Acids Res. 11:1475-1489.
Ding, C., M. Urabe, M. Bergoin, and R. M. Kotin. 2002. Biochemical characterization of Junonia coenia densovirus nonstructural protein NS-1. J Virol. 76:338-345.
Don, M., M. Soderlund-Venermo, F. Valent, A. Lahtinen, L. Hedman, M. Canciani, K. Hedman, and M. Korppi. 2010. Serologically verified human bocavirus pneumonia in children. Pediatr Pulmonol. 45:120-126.
Fasina, O. O., Y. Dong, and D J. Pintel. 2015. NP1 Protein of the Bocaparvovirus Minute Virus of Canines Controls Access to the Viral Capsid Genes via Its Role in RNA Processing. J.Virol. 90 1718-1728
Francois, A., M. Guilbaud, R. Awedikian, G. Chadeuf, P. Moullier, and A. Salvetti. 2005. The cellular TATA binding protein is required for rep-dependent replication of a minimal adeno-associated virus type 2 p5 element. J Virol. 79:11082-11094.
Garcia-Garcia, M. L., C. Calvo, A. Falcon, F. Pozo, P. Perez-Brena, J. M. De Cea, and I. Casas. 2010. Role of emerging respiratory viruses in children with severe acute wheezing. Pediatr. Pulmonol. 45:585-591.
Gendrel, D., R. Guedj, C. Pons-Catalano, A. Emirian, J. Raymond, F. Rozenberg, and P. Lebon. 2007. Human bocavirus in children with acute asthma. Clin. Infect Dis. 45:404-405.
Guan, W, S. Wong, N. Zhi, and J. Qiu. 2009. The genome of human parvovirus B19 virus can replicate in non-permissive cells with the help of adenovirus genes and produces infectious virus J. Virol. 83.9541-9553.
Hendrie, P. C., R. K. Hirata, and D. W. Russell. 2003. Chromosomal integration and homologous gene targeting by replication-incompetent vectors based on the autonomous parvovirus minute virus of mice. J Virol. 77:13136-13145.
Hickman, A. B., D. R. Ronning, Z. N. Perez, R. M. Kotin, and F. Dyda. 2004. The nuclease domain of adeno-associated virus rep coordinates replication initiation using two distinct DNA recognition interfaces. Mol.Cell. 13:403-414.
Huang, Q., X. Deng, Z. Yan, F. Cheng, Y. Luo, W. Shen, D. C. Lei-Butters, A. Y. Chen, Y. Li, L. Tang, M. Soderlund-Venermo, J. F. Engelhardt, and J. Qiu. 2012 Establishment of a reverse genetics system for studying human bocavirus in human airway epithelia. PLoS.Pathog. 8: e1002899.
Ihalainen, T. O., E. A. Niskanen, J. Jylhava, T. Turpeinen, J. Rinne, J. Timonen, and M. Vihinen-Ranta. 2007. Dynamics and interactions of parvoviral NS1 protein in the nucleus. Cell Microbiol. 9:1946-1959.
Jartti, T., K. Hedman, L. Jartti, O. Ruuskanen, T. Allander, and M. Soderlund-Venermo. 2011. Human bocavirus-the first 5 years. Rev. Med. Virol. 22:46-64.
Kahn, J. 2008. Human bocavirus: clinical significance and implications. Curr.Opin. Pediatr. 20:62-66.
Kapoor, A, N. Mehta, F. Esper, M. Polisak-Prijatelj, P. L. Quan, N. Qaisar, E. Delwart, and W. I Lipkin. 2010. Identification and characterization of a new bocavirus species in gorillas. PLoS ONE 5: e11948.
Li, Q., Z. Zhang, Z. Zheng, X. Ke, H. Luo, Q. Hu, and H. Wang. 2013. Identification and characterization of complex dual nuclear localization signals in human bocavirus NP1: identification and characterization of complex dual nuclear localization signals in human bocavirus NP1. J Gen. Virol. 94:1335-1342.
Luo, Y., S. Lou, X. Deng. Z. Liu, Y. Li, S. Kleiboeker, and J. Qiu. 2011. Parvovirus B19 infection of human primary erythroid progenitor cells triggers ATR-Chk1 signaling, which promotes B19 virus replication. J. Virol. 85:8046-8055.
Luo, Y., X. Deng, F. Cheng, Y. Li, and J. Qiu 2013. SMC1-mediated intra-S phase arrest facilitates Bocavirus DNA replication. J. Virol. 87:4017-4032.
Maiti, R., G. H. Van Domselaar, H. Zhang, and D. S. Wishart. 2004. SuperPose: a simple server for sophisticated structural superposition. Nucleic Acids Res. 32: W590-W594.
Martin, E. T., J. Kuypers, J. P. McRoberts, J. A. Englund, and D. M. Zerr. 2015. Human Bocavirus-1 Primary Infection and Shedding in Infants. J. Infect. Dis. 212:516-524.
Mihaylov, I. S., S. F. Cotmore, and P. Tattersall. 2014. Complementation for an essential ancillary non-structural protein function across parvovirus genera. Virology. 468-470:226-237.
Nash, K., W. Chen, M. Salganik, and N. Muzyczka. 2009. Identification of cellular proteins that interact with the adeno-associated virus rep protein. J. Virol. 83:454-469.
Nony, P., J. Tessier, G. Chadeuf, P. Ward, A. Giraud, M. Dugast, R. M. Linden, P. Moullier, and A. Salvetti 2001. Novel cis-acting replication element in the adeno-associated virus type 2 genome is involved in amplification of integrated rep-cap sequences. J Virol. 75:9991-9994.
Qiu, J., F. Cheng. F. B. Johnson, and D. Pintel. 2007. The transcription profile of the bocavirus bovine parvovirus is unlike those of previously characterized parvoviruses. J. Virol. 81:12080-12085.
Ryan, J. H., S. Zolotukhin, and N. Muzyczka. 1996. Sequence requirements for binding of Rep68 to the adeno-associated virus terminal repeats. J Virol. 70.1542-1553.
Schildgen, O., A. Muller, T. Allander, I. M. Mackay, S. Volz, B. Kupfer, and A. Simon. 2008. Human bocavirus: passenger or pathogen in acute respiratory tract infections? Clin. Microbiol. Rev. 21:291-304.
Shen, W., X. Deng, W. Zou, F. Cheng, J. F. Engelhardt, Z. Yan, and J. Qiu. 2015. Identification and Functional Analysis of Novel Non-structural Proteins of Human Bocavirus 1. J. Virol. 89:10097-10109.
Smith, D. H., P Ward, and R. M. Linden. 1999. Comparative characterization of rep proteins from the helper-dependent adeno-associated virus type 2 and the autonomous goose parvovirus. J Virol 73:2930-2937.
Soderberg, O., M. Gullberg, M. Jarvius, K. Ridderstrale, K. J. Leuchowius, J. Jarvius, K. Wester, P. Hydbring, F. Bahram, L. G. Larsson, and U. Landegren. 2006. Direct observation of individual endogenous protein complexes in situ by proximity ligation. Nat. Methods. 3:995-1000.
Sukhu, L., O. Fasina, L. Burger, A. Rai, J. Qiu, and D. J. Pintel. 2013. Characterization of the nonstructural proteins of the bocavirus minute virus of canines. J Virol. 87:1098-1104.
Sun, Y., A. Y. Chen, F. Cheng, W. Guan, F. B. Johnson, and J. Qiu. 2009. Molecular characterization of infectious clones of the minute virus of canines reveals unique features of bocaviruses J. Virol. 83:3956-3967.
Tam, P. and C. R. Astell. 1993. Replication of minute virus of mice minigenomes: novel replication elements required for MVM DNA replication. Virology. 193:812-824.
Tewary, S. K., H. Zhao, W. Shen, J. Qiu, and L. Tang. 2013. Structure of the NS1 protein N-terminal origin-recognition/nickase domain from the emerging human bocavirus. J. Virol. 87.11487-11494.
Tewary, S. K., H. Zhao, X. Deng. J. Qiu, and L. Tang. 2014. The human parvovirus B19 non-structural protein 1 N-terminal domain specifically binds to the origin of replication in the viral DNA. Virology. 449:297-303.
Tewary, S. K., L. Liang, Z. Lin, A. Lynn, S. F. Cotmore, P. Tattersall, H. Zhao, and L. Tang. 2015. Structures of minute virus of mice replication initiator protein N-terminal domain: Insights into DNA nicking and origin binding. Virology 476 61-71.
Tullis, G. E. and T. Shenk. 2000. Efficient replication of adeno-associated virus type 2 vectors. a cis-acting element outside of the terminal repeats and a minimal size. J Virol. 74:11511-11521.
Wang, X. S. and A. Srivastava. 1997. A novel terminal resolution-like site in the adeno-associated virus type 2 genome. J Virol. 71:1140-1146.
Ward, P. 2006. Replication of adeno-associated virus DNA, p. 189-211. In: J. Kerr, Cotmore SF. M. E. Bloom, M. E. Linden, and C. R. Parrish (eds.), The parvoviruses. Hodder Arnold, London.
Yan, Z., N. W. Keiser, Y. Song, X. Deng, F. Cheng, J. Qiu, and J. F. Engelhardt. 2013. A novel chimeric adenoassociated virus 2/human bocavirus 1 parvovirus vector efficiently transduces human airway epithelia. Mol. Ther. 21:2181-2194.
Zadori, Z., R. Stefancsik, T. Rauch, and J Kisary. 1995 Analysis of the complete nucleotide sequences of goose and muscovy duck parvoviruses indicates common ancestral origin with adeno-associated virus 2. Virology. 212:562-573.
Zhang. Y. 2008. I-TASSER server for protein 3D structure prediction. BMC.Bioinformatics. 9:40. doi: 10.1186/1471-2105-9-40.: 40-49.
Zou, W., F. Cheng. W. Shen, J. F. Engelhardt, Z. Yan, and J. Qiu. 2016. Nonstructural Protein NP1 of Human Bocavirus 1 Plays a Critical Role in the Expression of Viral Capsid Proteins. J. Virol. February
24. pil: JVI.02964-15. [Epub ahead of print].

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. (canceled)

2. A method to express a transgene in a cell comprising introducing a composition comprising a recombinant parvovirus vector comprising a synthetic enhancer comprising a plurality of enhancer sequences operably linked to a promoter operably linked to a transgene to a eukaryotic cell so as to express the transgene in the cell.

3. The method of claim 2, wherein the cell is in a mammal.

4. The method of claim 2, wherein the expression of the transgene is enhanced by at least 2-, 5-, 10-, or 15-fold or more relative to a corresponding parvovirus vector that lacks the synthetic enhancer.

5. The method of claim 2, wherein the plurality of enhancer sequences has 2 up to 20, 2 up to 15 or 2 up to 10, of the enhancer sequences in tandem.

6. The method of claim 2, wherein the synthetic enhancer comprises F1, F5 or F10, the synthetic enhancer has at least one enhancer sequence with at least 80% nucleotide sequence identity to F1, F5 or F10, the synthetic enhancer has at least one enhancer sequence with at least 90% nucleotide sequence identity to F1, F5 or F10 or the synthetic enhancer has at least one enhancer sequence with at least 95% nucleotide sequence identity to F1, F5 or F10.

7. The method of claim 2, wherein the synthetic enhancer and promoter have at least 80%, 85%, 90%, 95%, 98%, 99% or more nucleotide sequence identity to one of SEQ ID NOs: 24-30.

8. The method of claim 2, wherein the synthetic enhancer and promoter have a sequence having at least 90% nucleotide sequence identity to SEQ ID NO:27.

9. The method of claim 8, wherein the synthetic enhancer and promoter have at least 95%, 98%, 99%, or more nucleotide sequence identity to SEQ ID NO:27.

10. The method of claim 9, wherein the synthetic enhancer and promoter comprise the sequence of SEQ ID NO:27.

11. The method of claim 2, wherein the recombinant parvovirus vector is a bocavirus or an adeno-associated virus vector.

12. The method of claim 11, wherein the recombinant parvovirus vector is an adeno-associated virus vector.

13. The method of claim 2, wherein the promoter is operably linked to an open reading frame that encodes a prophylactic or therapeutic gene product.

14. The method of claim 13, wherein the therapeutic gene product is cystic fibrosis transmembrane conductance regulator (CFTR) or CFTRΔR.

15. The method of claim 14, wherein the CFTR or CFTRAR is human CFTR or human CFTRΔR.

16. The method of claim 15, wherein the CFTRAR is human CFTRΔR.

17. A method to express a human CFTRAR transgene in a cell comprising introducing a composition comprising a recombinant parvovirus vector to a eukaryotic cell so as to express the human CFTRAR transgene in the cell, wherein the recombinant parvovirus vector comprises (i) an enhancer operably linked to a promoter, wherein the enhancer and promoter comprise the sequence of SEQ ID NO: 27, and (ii) the human CFTRΔR transgene operably linked to the promoter.
